(19) United States
(12) Patent Application Publication
(10) Pub. No.: US 2009/0197256 A1
(43) Pub. Date: Aug. 6, 2009

(54) Use of a fibroblast growth factor-binding protein for the treatment and diagnosis of diabetic wound healing problems

(75) Inventors: Andreas Goppelt, Vienna (AT); Michaela Bittner, Vienna (AT)

Correspondence Address: CLARK & ELBING LLP, 101 FEDERAL STREET, BOSTON, MA, 02110, US

(73) Assignee: Fraunhofer-Gesellschaft zur forderung der angewandten Forschung e.V., Munich (DE)

(21) Appl. No.: 12/012,676

(22) Filed: Feb. 5, 2008

Related U.S. Application Data
(63) Continuation of application No. 10/927,322, filed on Aug. 26, 2004, now abandoned.
Application No. 10/927,322 is a continuation-in-part of application No. PCT/EP03/02122, filed on Feb. 28, 2003.
(60) Provisional application No. 60/362,497, filed on Mar. 6, 2002.

(30) Foreign Application Priority Data
Feb. 28, 2002 (EP) 02004299.0

(51) Int. Cl. C12Q 1/68; G01N 33/53; G01N 33/68; C12Q 1/02

(52) U.S. Cl. 435/6; 436/501; 436/86; 435/29

(57) ABSTRACT

The present invention relates to the use of fibroblast growth factor-binding protein (FGF-BP) polypeptides, and functional variants of these polypeptides, respectively, or of nucleic acids encoding these polypeptides or variants, or of functional variants of these nucleic acids, and/or of a cell which is expressing an FGF-BP polypeptide or functional variants thereof, for treating, diagnosing and/or preventing wound healing disturbances which are characterized by a reduced quantity of FGF-BP, in particular diabetes-associated wounds, and also to the use of at least one FGF-BP polypeptide as depicted in SEQ ID No. 1 and SEQ ID No. 3 and/or of at least one FGF-BP-encoding nucleic acid as depicted in SEQ ID No. 2 and SEQ ID No. 4 and/or of antibodies or antibody fragments which are directed against an FGF-BP polypeptide which can be used in accordance with the invention, or functional variants thereof, and/or of a cell which is expressing an FGF-BP polypeptide or functional variants thereof, for identifying pharmacologically active substances which increase the activity of expression of FGF-BP.

This application is a Continuation of U.S. patent application Ser. No. 10/927,322, filed Aug. 26, 2004, which is a Continuation-in-Part of International Application No. PCT/EP2003/02122, filed Feb. 28, 2003, which claims benefit of European patent application 02004299.0, filed Feb. 28, 2002, and U.S. Provisional application 60/362,497, filed Mar. 6, 2002.

The present invention relates to the use of fibroblast growth factor-binding protein (FGF-BP) polypeptides and functional variants of these polypeptides, respectively, or of nucleic acids and functional variants thereof, respectively, encoding these polypeptides or variants, and/or of a cell which is expressing an FGF-BP polypeptide or functional variants thereof, for treating, diagnosing and/or preventing wound healing disturbances which are characterized by a reduced quantity of FGF-BP, in particular diabetes-associated wounds, and also to the use of at least one FGF-BP polypeptide as depicted in SEQ ID No. 1 and SEQ ID No. 3 and/or of at least one FGF-BP-encoding nucleic acid as depicted in SEQ ID No. 2 and SEQ ID No. 4 and/or of antibodies or antibody fragments which are directed against an FGF-BP polypeptide, or functional variants thereof, which can be used in accordance with the invention, and/or of a cell which is expressing an FGF-BP polypeptide or functional variants thereof, for identifying pharmacologically active substances which increase the activity or expression of FGF-BP.

The skin wounds of healthy patients normally heal without any problems. However, a large number of chronological and spatial changes in the cell composition of the skin are required in order to achieve complete healing of the tissue. This process can last up to two years and, in non-foetal tissue, is always associated with scar formation. This points to the enormous complexity of the wound healing process in the skin. In connection with wound healing, it is possible to distinguish the chronologically sequential, partially overlapping phases of coagulation, inflammation, proliferation and reorganization (The physiology of wound healing, 1998, Oxford Institute for Continuing Education). During coagulation, blood platelets aggregate, with these platelets releasing growth factors and coagulation factors. A fibrin matrix is formed, with this matrix enabling cell migration to take place in the wound. An inflammatory reaction then occurs approx. 5-7 days after the injury. In connection with this, various cell types, in particular neutrophilic granulocytes and monocytes, migrate into the wound and release mediators of the inflammatory reaction. The proliferation phase serves the purpose of regenerating the damaged tissue and restructuring the tissue which has been regenerated. The processes involved include, in particular, neovascularization, fibroblast proliferation and reepithelialization, brought about by the proliferation and differentiation of a very wide variety of skin cells and are regulated by a very wide variety of growth factors and/or combinations of these growth factors. For example, the vascularization is promoted by the growth factors bFGF, VEGF and TGF-beta 1 and 2, which are produced by ruptured endothelial cells and macrophages, respectively. A very wide variety of cell types are involved in the production of factors, such as EGF, TGF-alpha, bFGF, aFGF and KGF, which control the proliferation of keratinocytes. The fibroblasts in turn secrete several growth factors, e.g. PDGF and TGF-beta, which regulate the synthesis and deposition of components of the extracellular matrix (ECM), such as fibronectin, laminin, glycosoaminoglycans and collagen. During the reorganization of the tissue, the ECM components, in particular the collagen, are rearranged. As a result of the collagen being constantly broken down and resynthesized, it is possible for the reepithelialized wound to mature, and a flat scar is formed within two years. Once again, a large number of growth factors and chemoattractants are required for the co-ordinated restructuring of the tissue. Thus, interleukin 1, TNF-beta and interferon-gamma exert an effect on the secretion of the ECM components. Furthermore, TGF-beta, PDGF, VEGF and FGF are essential for the reorganization.

This precisely co-ordinated, complex interplay of the physiological processes which occur in association with wound healing makes it clear that a very wide variety of disturbances can lead to the wound healing process being impaired. Some of these factors are, for example, age, immune diseases, nutritional deficiencies, zinc deficiency, innervation disturbances, alcohol abuse and genetic defects. Severe impairments of the wound healing process can then lead to chronic wounds and finally to ulcers. However, the therapies which have so far been developed for being able to intervene in chronic wound healing disturbances offer little satisfaction. Established forms of therapy are restricted to the physical support of wound healing (e.g. dressings, compresses and gels), to the curettage of necrotic tissue and to the transplantation of skin tissues, cultured skin cells and/or matrix proteins.

Since the above-listed growth factors appear to play a crucial role in achieving successful wound healing and an aesthetic result, the growth factors have in the past constituted an interesting subject for developing therapies. Since, however, the correct interplay of the numerous growth factors is influenced by a multiplicity of factors, such as the quantity, the spatial and chronological distribution, and the combination of growth factors, this approach turns out to be extremely complicated. Thus, while growth factors have in recent years been tested for their ability to improve wound healing, they have not so far been found to effect a significant improvement in the conventional therapy. The only one to be approved is PDGF-BB, which is used for treating diabetic foot ulcers.

However, an aspect of wound healing disturbances which has so far been disregarded is that of the pathogenic backgrounds which underlie the respective disturbances and which require different therapies due to the molecular causes being different. Thus, venous ulcers are customarily investigated from the molecular point of view and regarded as being representative of chronic wounds and/or wounds which heal poorly. However, very different disorders, with differing pathogenic backgrounds, come under the umbrella of chronic skin wounds. In general, the most frequent representatives of chronic skin wounds are distinguished as being diabetic ulcers, venous ulcers, neuropathic ulcers, arterial ulcers and decubitus ulcers. Decubitus ulcers are very deep wounds which are caused by the effect of pressure over relatively long periods and which are accompanied by necrosis, infection and maceration of the tissue. By contrast, venous ulcers, which are brought about by venous stasis, are if anything superficial. On the other hand, arterial ulcers are frequently caused by arterial occlusion diseases. Diabetic ulcers are in turn ulcers which frequently occur in diabetes patients. In addition to a large number of diseases, the late complications of diabetes also encompass characteristic changes in the skin such as frequent infections, trophic disturbances and necrobiosis lipoidica. These changes can develop into poorly healing ulcers. Thus, for example, 25% of patients with type II diabetes frequently suffer from chronic ulcers (e.g. “diabetic foot”), about half of which require elaborate treatments in hospital and in the end heal poorly despite this. Diabetic foot alone gives rise to more hospital admissions than does any other complication associated with diabetes. The number of these cases associated with diabetes type I and type II is increasing and represents approx. 2.5% of all admissions to hospital.

The object of the present invention is therefore to find a novel active compound which significantly improves, in particular, the healing, diagnosis and/or prevention of wound healing disturbances, in particular of diabetes-associated wounds which heal poorly.

Surprisingly, it has now been possible to demonstrate that, in contrast to other ulcer patients, diabetic patients are selectively susceptible to exhibiting a decreased quantity of FGF-BP. The study of expression in association with normal wound healing which is described in Example 1 demonstrates that an increased expression of FGF-BP is required for normal wound healing. This also becomes evident when a comparison is made with venous ulcers, in which a permanent slight increase in the expression of FGF-BP is observed. By contrast, in diabetic patients, there is selectively a predisposition to poorer wound healing which is characterized by a decreased expression of FGF-BP. This makes it possible to differentiate diabetic ulcers relatively well from normal wounds and other ulcers.

Equally surprising it has been possible to demonstrate that not only diabetes-associated wounds characterized by reduced expression of FGF-BP are able to be treated well by FGF-BP, but this also applies generally to all diabetes-associated wounds, therefore also to these, whose FGF-BP expression level is not reduced. This has the advantage that the FGF-status of each wound needs not to be determined before the start of treatment as each diabetes-associated wound can be treated by FGF-BP. The objects of invention described in the following description including all embodiments affect therefore not only wounds, in particular diabetes-associated wounds characterized by a reduced quantity of FGF-BP, but all diabetes-associated wounds independent of their expressed FGF-BP quantity.

These results, which are precisely explained in the examples, are summarized below:

Biopsies from human diabetic ulcers contained the mRNA encoding FGF-BP in markedly lower concentrations than did biopsies of venous ulcers or biopsies of normally healing wounds. This provides evidence that diabetes-associated wound healing disturbances, in particular, are distinguished by a decreased expression of FGF-BP which is specific for these diseases. Identifying a patient group which unexpectedly exhibits a deficiency in FGF-BP creates the possibility of employing FGF-BP, nucleic acid(s) encoding the protein, antibodies directed against a polypeptide, or functional variants thereof, which can be used in accordance with the invention, and/or cells expressing the protein, to achieve a successful and effective therapy and diagnosis of diabetes-associated wounds. It was also possible to reproduce these results in mice, thereby creating a suitable animal model for developing therapies. Thus, a comparison of wound healing between normal mice and diabetic mice, which exhibited poorer wound healing, demonstrates that, in diabetic mice, FGF-BP is expressed more weakly, on average by a factor of 2.8, during wound healing than it is in intact skin.

The object is therefore achieved, in accordance with the invention, by using one or more FGF-BP polypeptides or nucleic acids encoding these polypeptides.

The present invention therefore relates to the use of at least one FGF-BP polypeptide as depicted in SEQ ID No. 1 and SEQ ID No. 3, or functional variants thereof, or of the nucleic acid(s) as depicted in SEQ ID No. 2 and SEQ ID No. 4, or functional variants thereof, which encode the polypeptides or their variants, or of antibodies or antibody fragments which are directed against a polypeptide, or functional variants thereof, which can be used in accordance with the invention, or of a cell which is expressing FGF-BP, for treating, diagnosing and/or preventing wound healing disturbances which are characterized by a reduced quantity of FGF-BP, in particular diabetes-associated wounds. A preferred diabetes-associated wound which heals poorly is the diabetic ulcer.

The present invention also relates to the use of at least one FGF-BP polypeptide as depicted in SEQ ID No. 1 and 3, or functional variants thereof, and/or of at least one FGF-BP-encoding nucleic acid as depicted in SEQ ID No. 2 and 4, or functional variants thereof, or of antibodies or antibody fragments which are directed against an FGF-BP polypeptide, or functional variants thereof, which can be used in accordance with the invention, and/or of at least one FGF-BP polypeptide-expressing cell, for identifying pharmacologically active substances which increase the function and/or expression of the FGF-BP polypeptide and/or of an FGF-BP polypeptide-encoding nucleic acid for the purpose of treating and/or preventing skin diseases which are characterized by a reduced quantity of FGF-BP, in particular diabetic wound healing disturbances. In this connection, a preferred use of the said substances and cells is that of preparing a test for finding substances which are pharmacologically active in connection with skin diseases which are characterized by a reduced quantity of FGF-BP, in particular diabetic wound healing disturbances.

Human FGF-BP is a polypeptide which has a molecular weight of 17 kD which can exist as a monomer and also as a homodimer. It was originally isolated from culture medium which had been conditioned with human epidermoid carcinoma cells (Wu et al. (1991) J. Biol. Chem. 5:16778-16785). Furthermore, a marked increase in the expression of FGF-BP has been observed, in particular, in early stages of spongiform carcinomas, in normal and immortalized human keratinocytes, and also in renal tubules in association with renal diseases such as HIV-associated nephropathy or haemolytic uraemic syndrome (HUS) (Liu et al. (2001) Kidney Int., 59:1717-1728).

FGF-BP is known to bind FGF-1 (aFGF) and FGF-2 (bFGF) non-covalently and reversibly (Wu et al. (1991) J. Biol. Chem. 5:16778-16785). FGF-1 and FGF-2 belong to the family of the FGF proteins, to which more than 20 different proteins belong and which are expressed in a very wide variety of tissues. FGFs are what are termed translocating proteins, which are secreted independently of the normal secretory pathway and bind to the extracellular matrix, as a result of which they are protected from degradation, on the one hand, and, on the other hand, form a local reservoir of inactive protein which permits strict spatial regulation of the FGF signal pathway. The release of FGF from the extracellular matrix enables the FGF to bind to its receptor. Thus, bFGF, for example, is secreted by a very wide variety of cell types, such as keratinocytes, fibroblasts, endothelial cells and macrophages, and, for its part, exerts chemotactic and mitogenic effects in either an autocrine or paracrine manner (Powers et al. (2000) Endocr. Relat. Cancer 7:165-197).

At present, two mechanisms are known for releasing the FGFs. The first mechanism involves enzymic cleavage by plasmin or heparanase, the second involves binding to FGF-BP as carrier protein. The presence of FGF-BP can consequently contribute to increasing the activity of the FGFs. Such a stimulatory effect has thus far been demonstrated for angiogenesis and proliferation exclusively in connection with the origin and growth of tumours. Thus, it has been demonstrated that recombinant FGF-BP can directly, without cofactors, bind to bFGF and, at the same time, reduce the affinity of the bFGF for heparin and stimulate the proliferation of 3T3 fibroblasts and endothelial cells. Consequently, FGF-BP is highly involved in the origin and progression of tumours and has been exclusively discussed in this connection. This is additionally confirmed by the observed synergistic effect of bFGF and FGF-BP on angiogenesis in the chorioallantois membrane assay. It was possible to block this paracrine effect completely using antibodies directed against FGF2 (Tassi et al. (2001) J. Biol. Chem. 276:40247-53; Aigner et al. (2001) Int. J. Cancer 92:510-517). Furthermore, the in vitro ablation of FGF-BP by means of ribozymes in colon carcinoma cells led to a decrease in the release of biologically active bFGF (FGF2) and to a reduction in tumour growth and angiogenesis in xenotransplanted tumours (Czubayko et al. (1997) Nat. Med. 3:1083-1084; WO 99/15703). The tumour-promoting effect of FGF-BP was also reflected in the regulation of the transcripts. Thus, FGF-BP transcripts were downregulated by factors, such as retinoic acid, which control differentiation (Liaudet-Coopman et al. (1996) J. Biol. Chem. 271:21303-21308). By contrast, FGF-BP transcripts were upregulated by tumour growth-promoting factors, such as 12-O-tetradecanoylphorbol-13-acetate (TPA), serum and what is termed the epidermal growth factor (EGF) (see, for example, Harris et al. (1998) J. Biol. Chem. 273:19130-19139).

The investigations carried out on mice have so far shown that FGF-BP stimulates the function of the FGFs in skin during embryogenesis and that FGF-BP is evidently downregulated in adult skin. It is only in the early stages of carcinogenesis that a powerful reexpression of FGF-BP is observed in the skin in vivo and in vitro (Kurtz et al. (1997) Oncogene 14:2671-2681). Interestingly, however, no connection between FGF-BP and the release of FGFs from the extracellular matrix, for the purpose of mediating proliferation and angiogenesis during disturbed wound healing processes, has so far been demonstrated or suggested, even though aFGF and bFGF have been demonstrated to have a wound healing-promoting effect in a very wide variety of animal models (Quirinia & Viidik et al. (1998) Scand. J. Plast. Reconstr. Surg. Hand. Surg. 32:9-18; Fu et al. (1998) Lancet 352:1661-4; Okumura et al. (1996) Biol. Pharm. Bull. 19:530-5; Mellin et al. (1995) J. Invest. Dermatol. 104:850-5). On the contrary, according to the present state of the art, FGFs which play a role in wound healing are in the main released non-specifically as a result of mechanical injury to the cells (McNeil et al. (1989) J. Cell Biol. 109:811-22).

However, the fact that the quantity of FGF-BP is surprisingly reduced selectively in diabetes-associated wounds leads to a completely new situation: the poorer wound healing seen in diabetic patients is connected to the inadequate local release of the existing reserves of FGF. Consequently, the present invention contributes to overcoming this technical prejudice, i.e. that the poorer wound healing seen in diabetic patients is the result of a decreased availability of FGF, and opens up the possibility of significantly increasing the activity of FGFs in wound healing disturbances, in particular in diabetes-associated wounds, by means of administering FGF-BP locally.

Since an increased expression of FGF-BP, and the regulation of FGF which is associated therewith, has previously only been connected experimentally to the origin and progression of tumour diseases, the possibility of administering such a protein and/or nucleic acid for the purpose of treating diseases has not so far been considered at all. However, the surprising identification of wound healing disturbances, in particular of poorly healing, diabetes-associated wounds, which are characterized by a reduced quantity of FGF-BP offers the possibility, for the first time, of employing FGF-BP, (a) nucleic acid(s) encoding this protein, antibodies which are directed against FGF-BP, and/or cells which are expressing this protein, for treating, diagnosing and/or preventing these wound healing disturbances.

Wound healing, and its pathological disturbances within the meaning of the invention, are to be distinguished from skin diseases which are accompanied by deranged cell development and cell differentiation, in particular skin cancer. In the case of the latter disease, individual cells are transformed and, as a result, these cells begin to proliferate in an uncontrolled autonomous manner, i.e. in isolation from interactions with other cell types, and, at the same time, pass on these pathological changes to their daughter cells. Consequently, it is a disease which is associated with a loss of interactions, for example of cell-cell adhesion, and of typical cellular properties. By contrast, wound healing disturbances within the meaning of the invention are due to disturbances of skin cells in their physiological context. Thus, the course of wound healing can be modulated by a very wide variety of endogenous and exogenous factors. Even small disturbances in the interactions between the different cell types of the dermis and epidermis themselves, or else interactions with other tissues and organs, such as the blood vascular system, the nervous system and the connected tissue, can lead to disturbed wound healing followed by scar formation. Furthermore, infections, ageing, diseases such as diabetes and immune diseases, and also vitamin deficiencies, can impair the wound healing process. Examples of disturbed wound healing are the wounds of diabetic patients and alcoholics, wounds which are infected with microorganisms, ischaemic wounds and the wounds of patients suffering from deficient blood supply and venous stasis. Poorly healing wounds which are particularly preferred are diabetic, neuropathic, venous and arterial ulcers, in particular diabetes-associated wounds.

The autonomous character of cancer diseases is also seen at the therapeutic level. Cancer can be treated surgically in the case of non-metastasizing tumours. While this physical treatment is possible since no interactions take place between the tumour cells and the surrounding cells and tissues, thereby ensuring that the patient can be healed simply by excising the tumour, it is not possible in the case of wound healing disturbances within the meaning of the invention; the pathological disturbances in the cell-cell and/or tissue-tissue interactions cannot be remedied by excising affected areas of the skin. The fact that the diseases which are being compared are diseases which are based on fundamentally different mechanisms becomes clear when the therapeutic approaches are compared. In the case of cancer diseases and diseases which are accompanied by deranged cell proliferation, the therapy is directed towards destroying rapidly growing cells, for example using cytostatic agents. These toxic substances prevent the growth of actively proliferating cells whereas cells which are in the G0 phase of the cell cycle are unaffected. By contrast, the treatment of wound healing disturbances within the meaning of the invention is aimed at modulating the interactions between the different cell types, for example by influencing the migration, proliferation and differentiation of individual cell types. Wound healing disturbances within the meaning of the invention cannot be healed by means of a general inactivation of proliferating cells. The methodical approach for identifying the nucleic acids which are used in accordance with the invention and which are involved in wound healing and its disturbances within the meaning of the invention differs markedly from methods which are suitable for identifying nucleic acids which are involved in the processes of cancer diseases. The latter nucleic acids can be identified by analysing the genes of the cancer-affected cell type which are expressed differentially. However, by contrast, the aim of the assay of the present invention is to identify genes, by comparing expression in diseased and healthy tissue biopsies, which are involved in the complex processes of wound healing and its pathological disturbances. This method would not be suitable for identifying genes which are relevant to cancer.

Within the meaning of the present invention, “wound healing” is to be understood as meaning the process of healing a mechanical injury to the skin, such as incision wounds, abrasion wounds or the chafing of the skin, for example as the result of continuous stress, for example decubitus or necrotic processes, for example necrobiosis lipoidica.

Examples of “wound healing disturbances” within the meaning of the present invention are wounds of diabetic patients and alcoholics, wounds which are infected with organisms or viruses, ischaemic wounds, wounds of patients suffering from arterial occlusion diseases or venous insufficiency, and scars, preferably over-shooting scars, in particular cheloids. Particularly preferred poorly healing wounds are diabetic, neuropathic, venous and arterial ulcers, in particular diabetes-associated wounds.

Within the meaning of the present invention, “diabetes-associated wounds” are to be understood as meaning the skin wounds of non-human mammals and humans suffering from diabetes mellitus. Examples of these skin wounds are ulcers caused by diabetes, for example ulcus cruris arteriosum or necrobiosis lipoidica.

Within the meaning of the present invention, “activity” is to be understood as meaning the interaction between FGF-BP polypeptides themselves (homodimers) or else between FGF-BP polypeptides and various other molecules, such as perlecan (a heparan sulphate proteoglycan), heparin and FGF family members, such as FGF-1 and FGF-2, and also the physiological and functional proliferation and differentiation changes in cells and/or cell formations which are elicited thereby. Examples of these activities are to be found in Mongiat et al. (2001), J. Biol. Chem. 276:10263-10271 and Tassi et al. (2001) J. Biol. Chem. 276:40247-53.

“Quantity of FGF-BP” is to be understood as meaning both FGF-BP polypeptides and FGF-BP coding nucleic acids, whereby both are encompassed or each of them.

Within the meaning of the present invention, a “reduced quantity of FGF-BP” is to be understood as meaning both a reduced expression of FGF-BP polypeptides and FGF-BP-encoding nucleic acids and a reduced activity of FGF-BP polypeptides. The term also encompasses FGF-polypeptides and FGF-BP coding nucleic acids together as well as each of them in a separate manner.

Within the meaning of the present invention, the term “functional variants” of a polypeptide encompasses polypeptides which, for example, are regulated like the polypeptides employed in accordance with the invention during skin diseases which are accompanied by decreased levels of FGF-BP.

Within the meaning of the present invention, “functional variants” are also polypeptides which exhibit a sequence homology, in particular a sequence identity, of approx. at least 70%, preferably of approx. at least 80%, in particular of approx. at least 90%, especially of approx. at least 95%, with the polypeptide having the amino acid sequence as depicted in one of SEQ ID No. 1 and SEQ ID No. 3. In this connection, “sequence identity” is understood as meaning the congruence (=% positives) between two sequences, which congruence is determined using BlastP 2.0.1, for example, in the case of polypeptide sequences and using BLASTN 2.0.14, for example, in the case of polynucleotide sequences, with the filter being set at =off and BLOSUM being 62 (Altschul et al. (1997) Nucleic Acids Res. 25:3389-3402).

Examples of these functional variants are consequently the polypeptides which are homologous with a peptide which can be used in accordance with the invention and which are derived from other organisms than humans and mice, respectively, preferably from non-human mammals, such as monkeys, pigs and rats. Other examples of functional variants are polypeptides which are encoded by different alleles of the gene in different individuals or in different organs in an organism. Other examples of functional variants are also, for example, polypeptides which are encoded by an FGF-BP-encoding nucleic acid and which are isolated from a tissue which is not skin-specific, e.g. embryonic tissue, but which possess the above-described functions after having been expressed in a cell which is involved in wound healing.

In order to establish whether a polypeptide is a functional variant of a polypeptide which can be used in accordance with the invention, functional tests can be employed to compare the activity of the polypeptide to be tested with the activity of a polypeptide which can be used in accordance with the invention. On condition that the polypeptide to be tested fulfils the requirements of a functional variant at the level of sequence identity, the polypeptide to be tested is then a functional variant if its activity in the functional test turns out to be similar or identical to that of the polypeptide which can be used in accordance with the invention.

These functional tests include, for example, the application of an expression vector which contains a nucleic acid encoding the polypeptide to be tested, or the application of the polypeptide to be tested itself (Example 4), or of an antibody directed against the polypeptide to be tested, or of an antisense oligonucleotide, to wounds. Following incubation, for example of an expression vector, a comparison is made of the further progress in the wound healing in dependence on the different expression vectors administered, i.e. expression vectors containing a nucleic acid encoding the polypeptide to be tested or a nucleic acid encoding the polypeptide which can be used in accordance with the invention, or an expression vector without an insert. These functional tests can, for example, also be applied to the activity of the polypeptide to be tested in the context of disturbances in wound healing, for example in the context of poorly healing, dexamethasone-treated or diabetic wounds in animals. Thus, the application of PDGF-A and PDGF-B polypeptides, for example, to the poorly healing wounds of rabbits resulted in comparable improvements in wound healing (Tyrone, J W et al. (2000) J. Surg. Res. 93:230-236).

Functional variants of the polypeptide can also be parts of the polypeptide used in accordance with the invention which are at least 6 amino acids in length, preferably at least 8 amino acids in length, in particular at least 12 amino acids in length. Functional variants also include N-terminal and/or C-terminal and/or internal deletions of the polypeptide used in accordance with the invention in the range of approx. 1-60, preferably of approx. 1-30, in particular of approx. 1-15, especially of approx. 1-5, amino acids. For example, the first amino acid methionine can be lacking without the function of the polypeptide being significantly altered.

In addition, the polypeptides which can be used in accordance with the invention can be characterized by the fact that they are prepared synthetically. Thus, the entire polypeptide, or parts thereof, can be synthesized, for example, by means of classical synthesis (Merrifield technique). Parts of the polypeptides according to the invention are suitable, in particular, for isolating antisera which can be used to screen suitable gene expression libraries in order, in this way, to obtain further functional variants of the polypeptide according to the invention.

Polypeptides which can be used in accordance with the invention can be prepared using well-known recombinant methods. In this connection, the polypeptide is prepared, for example, by expressing the nucleic acids according to SEQ ID No. 2 and 4, or functional variants thereof, in a suitable expression system using methods which are well known to the skilled person. These expression systems are explained below. Examples of suitable cells are the E. coli strains DHS, HB101 or BL21, the yeast strain Saccharomyces cerevisiae, the insect cell line lepidopteran, e.g. from Spodoptera frugiperda, or the animal cells COS, Vero, 293, HaCaT and HeLa, which are all generally available. Thus, the FGF-BP according to the invention has already been successfully expressed in Sf-9 insect cells ((Example 4) Tassi et al. (2001) J. Biol. Chem. 276:40247-53).

Furthermore, polypeptides which can be used in accordance with the invention can be isolated from an organism or from tissue or cells and used in accordance with the invention. Thus, it is possible, for example, to purify polypeptides which can be used in accordance with the invention from mammalian tissue, for example from skin or from body fluids, for example blood, serum, saliva, synovial fluid or wound fluid. Furthermore, cell lines can be prepared from cells which express the polypeptides which can be used in accordance with the invention, with it then being possible to use the cell lines for isolating polypeptides which can be used in accordance with the invention. For example, expression vectors which contain the nucleic acids which can be used in accordance with the invention can be transformed into skin cells, for example HaCaT cells. The expression can, for example, be constitutive or inducible. Thus, the FGF-BP protein according to the invention has already been successfully purified from medium conditioned by cell lines and from bovine mammary gland tissues (Wu et al. (1991) J. Biol. Chem. 266:16-778-85; Wang et al. (1998) Biochem. Mol. Biol. Int. 46:81-87; Lametsch et al. (2000) J. Biol. Chem. 275:19469-19474).

Another embodiment relates to the use of the FGF-BP polypeptides according to the invention, with the variants of SEQ ID No. 1 and/or 3 and SEQ ID No. 2 and/or 4 being, respectively, fusion proteins or nucleic acids which encode fusion proteins. Fusion proteins which can be used in accordance with the invention can be prepared, for example, by expressing nucleic acids which can be used in accordance with the invention in a suitable cell.

The fusion proteins either themselves already exhibit the function of a polypeptide of the invention or are only functional once the fusion moiety has been eliminated. This includes, in particular, fusion proteins containing a moiety of approx. 1-300, preferably approx. 1-200, in particular approx. 1-100, especially approx. 1-50, foreign amino acids. Examples of such peptide sequences are prokaryotic peptide sequences which can be derived, for example, from E. coli galactosidase. It is furthermore also possible to use viral peptide sequences, for example from the bacteriophage M13, in order, in this way, to generate fusion proteins for the phage-display method, which is known to the skilled person.

Other preferred examples of peptide sequences for fusion proteins which can be used in accordance with the invention are peptides which facilitate detection of the fusion proteins. These include, for example, green fluorescent protein (WO 95/07463) or functional variants thereof.

An additional polypeptide (“tag”) can be added on for the purpose of purifying the above-described proteins. Protein tags according to the invention make it possible, for example, for the polypeptides to be adsorbed, with high affinity, to a matrix, and for the matrix then to be washed stringently with suitable buffers without the complex being eluted to any significant extent, and for the adsorbed complex subsequently to be eluted selectively. Examples of the protein tags which are known to the skilled person are a (His)6 tag, a Myc tag, a FLAG tag, a haemagglutinin tag, a glutathione transferase (GST) tag, intein having an affinity chitin-binding tag or maltose-binding protein (MBP) tag. These protein tags can be located N-terminally, C-terminally and/or internally.

The term “coding nucleic acid” refers to a DNA sequence, as depicted in SEQ ID No. 2 and SEQ ID No. 4, which encodes an isolatable, functional polypeptide according to the invention, or a precursor thereof, for example possessing a signal sequence. The polypeptide can be encoded by a full-length sequence or any part of the coding sequence as long as the specific activity, for example the enzymic activity, is preserved.

It is known that changes in the sequences of the above-described nucleic acids can be present, for example as a result of the degeneracy of the genetic code, or that untranslated sequences can be appended, for example at the 5′ end and/or 3′ end of the nucleic acid, without their activity being significantly altered. It is also possible to carry out the modifications which are described in more detail below. This invention therefore also encompasses what are termed “variants” of the above-described nucleic acids.

“Variants” of the nucleic acids are to be understood as meaning all the DNA sequences which are complementary to a DNA sequence which hybridizes with the reference sequence under stringent conditions and which code for polypeptides which exhibit an activity which is essentially the same as that of the polypeptide encoded by the reference sequence.

“Stringent hybridization conditions” are to be understood as meaning those conditions in which the hybridization takes place, for example, at 60° C. in 2.5×SSC buffer, followed by several washing steps at 37° C. in a lower buffer concentration, and remains stable.

Variants of the nucleic acid can also be parts of the nucleic acid, which is used in accordance with the invention, having a length of at least 8 nucleotides, preferably having a length of at least 18 nucleotides, in particular having a length of at least 24 nucleotides, particularly preferably having at least 30 nucleotides, especially preferably having at least 42 nucleotides.

The nucleic acids which can be used in accordance with the invention are preferably DNA or RNA, preferably a DNA, particularly preferably a double-stranded DNA. Furthermore, the sequence of the nucleic acids can be characterized by the fact that it possesses at least one intron and/or a polyA sequence. The nucleic acids which are used in accordance with the invention can also be present in the form of their antisense sequence.

Furthermore, it is possible to use a nucleic acid which has been prepared synthetically for carrying out the invention. Thus, the nucleic acid which is used in accordance with the invention can, for example, be synthesized chemically, e.g. in accordance with the phosphotriester method, with the aid of the DNA sequences which are described in Table 1 and/or with the aid of the protein sequences which are likewise described in Table 1 and making use of the genetic code (see, for example, Uhlmann, E. & Peyman, A. (1990) Chemical Reviews 90:543-584).

Within the meaning of the present invention, “expression” is to be understood as meaning the quantity of FGF-BP polypeptides and/or FGF-BP-encoding nucleic acids which is present in a particular cell type.

The term “regulation” is to be understood as meaning, for example, the increase or decrease in the quantity of polypeptide or of nucleic acid encoding this quantity, with it being possible for this change to take place, for example, at the transcriptional, translational, posttranscriptional and posttrans-lational level.

In general, a double-stranded DNA is preferred for expressing the relevant gene, with the DNA region which encodes the polypeptide being particularly preferred. In eukaryotes, this region extends from the first start codon (ATG) which is located in a Kozak sequence (Kozak, 1987. Nucleic. Acids Res. 15:8125-48) to the next stop codon (TAG, TGA or TAA) which is located in the same reading frame as the ATG. In prokaryotes, this region begins with a conserved sequence of 6 nucleotides, i.e. what is termed a Shine-Dalgarno sequence (Shine & Dalgarno, 1975, Eur. J. Biochem. 57:221-30) which is located a few nucleotides upstream of the start codon.

Expression vectors can be prokaryotic or eukaryotic expression vectors. Examples of prokaryotic expression vectors for expression in E. coli are, for example, the pGEM vectors or pUC derivatives, while examples of eukaryotic expression vectors for expression in Saccharomyces cerevisiae are, for example, the vectors p426Met25 and p426GAL1 (Mumberg et al. (1994) Nucl. Acids Res. 22:5767-5768), and examples for expression in insect cells are, for example, the Baculovirus vectors, as disclosed in EP-B1-0 127 839 or EP-B1-0 549 721, and for expression in mammalian cells are, for example, the Rc/CMV and Rc/RSV vectors or SV40 vectors, all of which are available generally.

In general, the expression vectors also contain promoters which are suitable for the respective cell, such as the trp promoter for expression in E. coli (see, for example, EP-B1-0 154 133), the Met 25, GAL 1 or ADH2 promoter for expression in yeasts (Russel et al. (1983) J. Biol. Chem. 258:2674-2682; Mumberg, see above) and the baculovirus polyhedrin promoter for expression in insect cells (see, for example, EP-B1-01 127 839). Promoters which permit constitutive, regulatable, tissue-specific, cell cycle-specific or metabolism-specific expression in eukaryotic cells are suitable, for example, for expression in mammalian cells. Regulatable elements according to the present invention are promoters, activator sequences, enhancers, silencers and/or repressor sequences.

Examples of suitable elements which enable constitutive expression to take place in eukaryotes are promoters which are recognized by RNA polymerase III or viral promoters, CMV enhancers, CMV promoters, SV40 promoters or LTR promoters, e.g. from MMTV (mouse mammary tumour virus; Lee et al. (1981) Nature 214:228-232) and other viral promoter and activator sequences derived, for example, from HBV, HCV, HSV, HPV, EBV, HTLV or HIV.

Wound healing-relevant genes are preferably expressed under the control of tissue-specific promoters, with skin-specific promoters, such as the human K10 promoters (Bailleul et al. (1990) Cell 62:697-708), the human K14 promoter (Vassar et al. (1989) Proc. Natl. Acad. Sci. USA 86:1563-67) or the bovine cytokeratin IV promoter (Fuchs et al. (1988) The biology of wool and hair (eds.: G. E. Rogers et al.), pp. 287-309. Chapman and Hall, London/New York) being particularly preferred.

Other examples of elements which make it possible to achieve tissue-specific expression in eukaryotes are promoters or activator sequences from promoters or enhancers of those genes which encode proteins which are only expressed constitutively in particular cell types.

Examples of an element which makes it possible to achieve regulatable expression in eukaryotes is the tetracycline operator in combination with a corresponding repressor (Gossen M. et al. (1994) Curr. Opin. Biotechnol. 5:516-20).

Examples of regulatable elements which make it possible to achieve cell cycle-specific expression in eukaryotes are promoters of the following genes: cdc25A, cdc25B, cdc25C, cyclin A, cyclin E, cdc2, E2F-1 to E2F-5, B-myb or DHFR (Zwicker J. and Müller R. (1997) Trends Genet. 13, 3-6). The use of cell cycle-regulated promoters is particularly preferred in cases in which the expression of the polypeptides or nucleic acids used in accordance with the invention is to be restricted to proliferating cells.

Examples of regulatable elements which make it possible to achieve metabolism-specific expression in eukaryotes are promoters which are regulated by hypoxia, glucose deficiency, phosphate concentration or heat shock.

Examples of regulatable elements which simultaneously permit expression which is spatially and temporally limited are nucleic acids which encode a fusion between the sequence for the site-specific recombinase Cre and a modified oestrogen receptor under the control of a tissue-specific promoter. The resulting tissue-specific cytoplasmic fusion protein is able, as a result of the oestrogen-analogue tamoxifen being added, to translocate into the cell nucleus and generate recombinations which lead to altered gene expression (Feil et al. (1996) Proc. Natl. Acad. Sci. 93: 10887-90).

The FGF-BP-encoding nucleic acid as depicted in SEQ ID No. 2 and 4, or functional variants thereof, can be used in the form of an expression vector, in particular of a vector which is active in gene therapy, in order to enable the above-described nucleic acids to be introduced, by transfection, transformation or infection, into a eukaryotic or prokaryotic cell and consequently to enable the FGF-BP polypeptide to be expressed in this cell. In additional examples, the FGF-BP-encoding nucleic acid can be present in a shuttle vector, phagemid, cosmid or plasmid, or be present as part of a viral or non-viral vector. In this connection, the following viral vectors are particularly suitable: baculoviruses, vacciniaviruses, adenoviruses, adeno-associated viruses and herpes-viruses. Non-viral vectors which are particularly suitable in this connection are: virosomes, liposomes, cationic lipids and polylysine-conjugated DNA.

A vector which is active in gene therapy preferably contains wound-specific and skin-specific regulatory sequences, respectively, which are functionally linked to the above-described nucleic acid.

When the above-described nucleic acid is to be used for gene therapy, it is also advantageous if the part of the nucleic acid which encodes the polypeptide contains one or more non-coding sequences, including intron sequences, preferably between the promoter and the start codon of the polypeptide, and/or a polyA sequence, in particular the naturally occurring polyA sequence or an SV40 virus polyA sequence, in particular at the 3′ end of the gene, since this makes it possible to stabilize the mRNA (Example 3; Palmiter et al. (1991) Proc. Natl. Acad. Sci. USA 88:478-482; Jackson (1993) Cell 74:9-14).

Within the meaning of the present invention, a “gene therapy vector” is to be understood as meaning a nucleic acid into which a nucleic acid which can be used in accordance with the invention can be integrated and which is subsequently introduced into a cell and, in this cell, supplements or replaces the missing or defective expression of the nucleic acid according to the invention.

Examples of vectors which are active in gene therapy are viral vectors, for example adenoviral vectors or retroviral vectors (Lindemann et al. (1997), Mol. Med. 3:466-76; Springer et al. (1988) Mol. Cell. 2:549-58). Eukaryotic expression vectors are suitable for use in gene therapy in isolated form since, when applied topically, naked DNA is able to penetrate into skin cells (Hengge et al. (1996) J. Clin. Invest. 97:2911-6; Yu et al. (1999) J. Invest. Dermatol. 112:370-5).

Vectors which are active in gene therapy can also be obtained by complexing the above-described nucleic acids with liposomes since this makes it possible to achieve a very high efficiency of transfection, in particular of skin cells (Alexander and Akhurst, 1995, Hum. Mol. Genet. 4:2279-85). In lipofection, small, unilamellar vesicles composed of cationic lipids are prepared by ultrasonicating the liposome suspension. The DNA is bound ionically on the surface of the liposomes in a ratio which is such that a positive net charge remains and all the plasmid DNA is complexed by the liposomes. In addition to the DOTMA (1,2-dioleyloxypropyl-3-trimethylammonium bromide) and DOPE (dioleoylphosphatidylethanolamine) lipid mixtures employed by Felgner et al. (1987, see above), a large number of new lipid formulations have by now been synthesized and tested for their efficiency in transfecting a variety of cell lines (Behr et al. (1989) Proc. Natl. Acad. Sci. USA 86:6982-6986; Gao and Huang (1991), Biochim. Biophys. Acta 1189:195-203; Felgner et al. (1994) J. Biol. Chem. 2692550-2561). Examples of the new lipid formulations are DOTAP N-[1-(2,3-dioleoyloxy)propyl]-N,N,N-trimethylammonium methyl sulphate or DOGS (TRANSFECTAM; dioctadecylamidoglycylspermine). Auxiliary substances which increase the transfer of nucleic acids into the cell can, for example, be proteins or peptides which are bound to the DNA or synthetic peptide-DNA molecules which enable the nucleic acid to be transported into the nucleus of the cell (Schwartz et al. (1999) Gene Therapy 6:282; Brandén et al. (1999) Nature Biotech. 17:784). Auxiliary substances also include molecules which enable nucleic acids to be released into the cytoplasm of the cell (Planck et al. (1994) J. Biol. Chem. 269:12918; Kichler et al. (1997) Bioconj. Chem. 8:213) or, for example liposomes (Uhlmann and Peymann (1990), see above).

Another, particularly suitable form of gene therapy vectors can be obtained by applying the above-described nucleic acid to gold particles and firing these particles into tissue, preferably into the skin, or cells using what is termed a “gene gun” (Wang et al. (1999) J. Invest. Dermatol. 112:775-81, Tuting et al. (1998) J. Invest. Dermatol. 111:183-8).

Another embodiment of a vector which is active in gene therapy and which can be used in accordance with the invention can be prepared by introducing “naked” expression vectors into a biocompatible matrix, for example a collagen matrix. This matrix can be introduced into wounds in order to transfect the inwardly migrating cells with the expression vector and to express the polypeptides according to the invention in the cells (Goldstein and Banadio, U.S. Pat. No. 5,962,427).

The therapy of wound healing disturbances, in particular diabetes-associated wounds, can be effected in a conventional manner, for example using dressings, plasters, compresses or gels which contain the medicaments according to the invention. Thus, it is possible to administer the medicaments, which comprise suitable additives or auxiliary substances, such as physiological sodium chloride solution, demineralized water, stabilizers, proteinase inhibitors, gel formulations, such as white vaseline, low-viscosity paraffin and/or yellow wax, etc., topically and locally in order to exert an immediate and direct influence on the wound healing.

The medicaments according to the invention can furthermore be administered, where appropriate, in the form of liposome complexes or gold particle complexes, likewise topically and locally in the region of the wound. In addition, the treatment can be effected using a transdermal therapeutic system (TTS) which enables the medicaments which can be used in accordance with the invention to be released in a time-regulated manner. However, the treatment with the medicaments which can be used in accordance with the invention can also be effected using oral dosage forms, such as tablets or capsules, by way of the mucous membranes, for example of the nose or the oral cavity, or in the form of depots which are implanted under the skin. TTSs are disclosed, for example, in EP 0 944 398, EP 0 916 336, EP 0 889 723 and EP 0 852 493.

A medicament which comprises the above-described nucleic acids in naked form or in the form of one of the above-described vectors which are active in gene therapy, or in a form which is complexed with liposomes or gold particles, is especially suitable for use in gene therapy in humans. The pharmaceutical excipient is, for example, a physiological buffer solution, preferably having a pH of approx. 6.0-8.0, preferably of approx. 6.8-7.8, in particular of approx. 7.4, and/or an osmolarity of approx. 200-400 milliosmol/liter, preferably of approx. 290-310 milliosmol/liter. In addition, the pharmaceutical excipient can contain suitable stabilizers, such as nuclease inhibitors, preferably sequestering agents such as EDTA and/or other auxiliary substances which are known to the skilled person.

The above-described nucleic acids are customarily administered, if appropriate, in the form of the viral vectors described in more detail above, or as liposome complexes or gold particle complexes, topically and locally in the region of the wound.

A preferred embodiment of a medicament in accordance with the present invention is that of administering the polypeptide itself, together with suitable additives or auxiliary substances, such as physiological sodium chloride solution, demineralized water, stabilizers, proteinase inhibitors, gel formulations, such as white vaseline, low-viscosity paraffin and/or yellow wax, etc., in order to exert an immediate and direct effect on the wound healing (Example 4). This embodiment is particularly promising in the case of the present invention since the FGF-BP polypeptide is a secreted protein.

Another preferred embodiment of a medicament according to the present invention is that of using an antibody which is directed against a polypeptide which can be used in accordance with the invention and which increases or supports the activity of the FGF-BP. For example, such an antibody could exert a positive influence on the release of FGF and contribute to treating and preventing wound healing disturbances which are characterized by a reduced quantity of FGF-BP.

Another preferred embodiment of a medicament according to the present invention is that of transplanting autologous or allogenic cells which express and secrete the FGF-BP polypeptide according to the invention using a suitable carrier material, for example what are termed microcarriers, which consist of biocompatible materials, such as a dextran matrix (U.S. Pat. No. 5,980,888).

Another preferred embodiment of the present invention is that of using a cell which is transformed with a vector which can be used in accordance with the invention, or a knock-out gene construct for the purpose of diagnosing and/or preventing wound healing disturbances, in particular diabetes-associated wounds, and for the purpose of identifying pharmacologically active substances. Cells can be either prokaryotic or eukaryotic cells. Examples of prokaryotic cells are E. coli and of eukaryotic cells are Saccharomyces cerevisiae or insect cells.

Cells which express an FGF-BP polypeptide according to the invention, or functional variants thereof, or antibodies or antibody fragments which are directed against FGF-BP polypeptides according to the invention, or functional variants thereof, are, for example, human, non-embryonic, autologous or allogenic cells. The said cells are preferably skin cells, in particular keratinocytes, fibroblasts or endothelial cells.

A particularly preferred transformed cell which can be used in accordance with the invention is a transgenic embryonic non-human stem cell which is characterized by the fact that it contains at least one knock-out gene construct which can be used in accordance with the invention and/or at least one expression cassette which can be used in accordance with the invention, as previously described. Methods for trans-forming cells and/or stem cells are well known to the skilled person and include electroporation or microinjection, for example.

Another preferred embodiment of a medicament in accordance with the present invention relates to the use of at least one FGF-BP polypeptide as depicted in SEQ ID No. 1 and 3, or functional variants thereof, and/or of at least one FGF-BP-encoding nucleic acid as depicted in SEQ ID No. 2 and 4, or functional variants thereof, or of antibodies or antibody fragments which are directed against an FGF-BP polypeptide which can be used in accordance with the invention, or functional variants thereof, and/or of at least one cell which is expressing an FGF-BP polypeptide, for identifying pharmacologically active substances which increase the function and/or the expression of the FGF-BP polypeptide and/or of an FGF-BP polypeptide-encoding nucleic acid for the purpose of treating and/or preventing skin diseases which are characterized by a reduced quantity of FGF-BP. The said pharmacologically active substances can be organic or inorganic substances which modulate, in particular activate, the activity and/or expression of the FGF-BP protein and/or DNA. The use of these organic or inorganic pharmacologically active substances as medicaments can be effected by means of application, as described above. The examples of such activating substances are to be found in Harris et al. (2000) J. Biol. Chem. 275:10802-10811.

The present invention furthermore relates to the use of at least one FGF-BP polypeptide as depicted in SEQ ID No. 1 and 3, or of a functional variant thereof, and/or of at least one FGF-BP-encoding nucleic acid as depicted in SEQ ID No. 2 and 4, or functional variants thereof, or of antibodies or antibody fragments which are directed against an FGF-BP polypeptide which can be used in accordance with the invention, or functional variants thereof, and/or of at least one cell which is expressing an FGF-BP polypeptide, where appropriate combined or together with suitable additives and auxiliary substances, for producing a test for finding substances which are pharmacologically active in connection with skin diseases which are characterized by a reduced quantity of FGF-BP, in particular diabetes-associated wounds.

Within the meaning of the present invention, the term “pharmacologically active substances” is to be understood as meaning all those molecules, compounds and/or compositions and substance mixtures which can interact, under suitable conditions, with the previously described nucleic acids, polypeptides, antibodies or antibody fragments, where appropriate together with suitable additives and auxiliary substances. Possible pharmacologically active substances are simple chemical organic or inorganic molecules or compounds, the term can also encompass nucleic acids, peptides and proteins or complexes thereof. As a result of their interaction, the pharmacologically active substances can exert an effect, in vivo or in vitro, on the function(s) of the nucleic acids, polypeptides or antibodies, or else only bind to the previously described nucleic acids, polypeptides, antibodies or antibody fragments, or enter into other interactions with them in a covalent or non-covalent manner. Examples of pharmacologically active substances are organic molecules which are derived from substance libraries which have been tested for their pharmacological activity.

According to the present invention, preference is given to those pharmacologically active substances which act in an activating manner.

In one embodiment, the tests for finding pharmacologically active substances of the FGF-BP according to the invention encompass assays which exert an influence on the expression of genes and which are well known to the skilled person (see, for example, Sivaraja et al. (2001) U.S. Pat. No. 6,183,956). Thus, cells which express FGF-BP, for example skin cells, can be cultured as a test system for analysing gene expression in vitro, with preference being given to keratinocytes, fibroblasts or endothelial cells. In this connection, the human keratinocyte cell line HaCaT, which is available generally, is a possible test system. Gene expression is analysed, for example, at the level of the mRNA or the proteins. In this analysis, the quantity of FGF-BP mRNA and/or protein is measured after adding one or more substances to the cell culture and compared with the corresponding quantity in a control culture. This takes place, for example, by means of hybridizing an antisense probe with the mRNA, which is present in the lysate of the cells, for an FGF-BP according to the invention. In this connection, the hybridization can be quantified, for example, by a specific antibody being bound to the mRNA/probe complex (see Stuart & Frank, 1998, U.S. Pat. No. 4,732,847). In this context it is possible to carry out this analysis as a high-throughput method and to analyse a very large number of substances for their suitability as modulators of FGF-BP expression (Sivaraja et al. (2001) U.S. Pat. No. 6,183,956). In this connection, the substances to be analysed can be taken from substance libraries (see, for example, DE 19816414, DE 19619373), which can contain several thousand substances, which are frequently very heterogeneous. Alternatively, the total RNA or total mRNA can first of all be isolated from cells and the absolute quantity, or the relative proportion, of the FGF-BP mRNA can then be determined, for example, by means of the quantitative RT-PCR (see EP 0 200 362; Wittwer, 1997, Bio Techniques 22:130-138; Morrison et al. (1998) Bio Techniques 24:954-62) or the RNase protection assay (see, for example, Sambrook et al. (1989) Molecular Cloning: A Laboratory Manual, Cold Spring Harbor Laboratory, Cold Spring Harbor Laboratory Press, New York, Chapter 7; EP 0 063 879). Another possibility is that of analysing the quantity of protein in the cell lysate using antibodies which recognize FGF-BP specifically. In this case, the method can be quantified, for example, using an ELISA or a Western blot, which are well known. In order to compare the specificity of the substances with regard to their effect on the expression of FGF-BP, the influence of the substances on the expression of FGF-BP can be compared with their influence on the expression of other genes, for example metabolism genes such as GAPDH or cyclophilin. Assays which are suitable for identifying substances which exert as little influence as possible on the expression of one or more control genes, such as GAPDH or cyclophilin, are particularly to be preferred (see, for example, WO 00/05416).

Another embodiment of the present invention relates to the use of FGF-BP polypeptides for identifying, with the aid of screening methods, pharmacologically active substances which increase the activity of FGF-BP. Thus, there is, for example, what is termed the “two-hybrid system” (Fields and Sternglanz, 1994, Trends in Genetics, 10, 286-292; Colas and Brent, 1998 TIBTECH, 16, 355-363). In this test, cells are transformed with expression vectors which are expressing fusion proteins consisting of the polypeptide according to the invention and a DNA-binding domain of a transcription factor such as Gal4 or LexA. The transformed cells additionally contain a reporter gene whose promoter contains binding sites for the corresponding DNA-binding domain. By transforming with another expression vector which is expressing a second fusion protein consisting of a known or unknown polypeptide and an activation domain, for example from Gal4 or herpes simplex virus VP16, the expression of the reporter gene can be greatly increased if the second fusion protein interacts with the polypeptide according to the invention. This increase in expression can be exploited for identifying novel pharmacologically active substances, for example by preparing a cDNA library from regenerating tissue for the purpose of constructing the second fusion protein. Furthermore, this test system can be used for screening for substances which inhibit an interaction between the polypeptide according to the invention and an interacter. Such substances decrease the expression of the reporter gene in cells which are expressing fusion proteins consisting of the polypeptide according to the invention and the pharmacologically active substance (Vidal and Endoh, 1999, Trends in Biotechnology, 17:374-81). In this way, it is possible rapidly to identify novel active compounds which can be used for treating disturbances of regenerative processes. The successful use of the FGF-BP according to the invention in two-hybrid systems has already been demonstrated by “fishing” FGF-BP from a keratinocyte library using perlecan as the “bait” (Mongiat et al., 2001, J. Biol. Chem. 276:10263-10271).

A preferred embodiment of these test systems which can be used in accordance with the invention is that of using, for example, HaCaT cells, which are available generally, and the expression vector pCMV4 (Anderson et al. (1989) J. Biol. Chem. 264:8222-9). In this method, an FGF-BP-encoding nucleic acid can be integrated into the expression vectors either in the sense orientation or in the antisense orientation such that the functional concentration of mRNA of the corresponding genes in the cells is either increased or else decreased due to hybridization with the antisense RNA. Following transformation, and selection of stable transformants, the cells in culture generally exhibit a proliferation, migration and/or differentiation behaviour which is altered as compared with that of control cells. This in vitro behaviour is frequently correlated with the function of the corresponding genes in regenerative processes in the body (Yu et al. (1997) Arch. Dermatol. Res. 289:352-9; Mils et al. (1997) Oncogene 14:15555-61; Charvat et al. (1998) Exp. Dermatol. 7:184-90; Werner (1998) Cytokine Growth Factor Rev. 9:153-65; Mythily et al. (1999) J. Gen. Virol. 80:1707-13) and can be detected using tests which can be carried out simply and rapidly such that it is possible, based on this, to construct test systems for pharmacologically active substances. Thus, the proliferation behaviour of cells can be very rapidly detected by, for example, incorporating labelled nucleotides into the DNA of the cells (see, for example, Savino and Dardenne, 1985, J. Immunol. Methods 85:221-6; Perros and Weightman, 1991, Cell Prolif. 24:517-23; de Fries and Mitsuhashi, 1995, J. Clin. Lab. Anal. 9:89-95), by staining the cells with specific dyes (Schulz et al. (1994) J. Immunol. Methods 167:1-13) or by means of the immunological methods (Frahm et al. (1998) J. Immunol. Methods 211:43-50). Migration can be detected simply by means of the migration index test (Charvat et al., see above) and comparable test systems (Benestad et al. (1987) Cell Tissue Kinet. 20:109-19, Junger et al. (1993) J. Immunol. Methods 160:73-9). Examples of suitable differentiation markers are keratin 6, and 14 as well as loricrin and involucrin (Rosenthal et al. (1992) J. Invest. Dermatol. 98:343-50), whose expression can readily be detected, for example, using generally available antibodies. The changes, which are identified in this way, in the activities of the cells, as compared with a controlled experiment in which cells have, for example, been transfected with an empty expression vector, can be used for investigating the efficiency of modulators.

In addition, a test system can be based on at least one FGF-BP polypeptide which can be used in accordance with the invention, or functional variants thereof, or nucleic acids encoding these polypeptides or variants thereof, or antibodies or antibody fragments which are directed against polypeptides which can be used in accordance with the invention, or functional variants thereof, being bound to a solid phase and at least one substance being examined for its pharmacological activity, for example binding or change in the conformation. Suitable systems, such as affinity chromatography and fluorescence spectroscopy, are known to the skilled person. Thus, binding between an FGF-BP according to the invention and heparin has already been successfully demonstrated using heparin-copper affinity chromatography (Wang et al. (1998) Biochem. Mol. Biol. Int. 46:81-87).

The solid phase-bound polypeptides, which can be used in accordance with the invention, or functional variants thereof, or nucleic acids encoding these polypeptides or a variant thereof, or antibodies or antibody fragments which are directed against a polypeptide which can be used in accordance with the invention, or a functional variant thereof, can also be part of an array. Methods for preparing such arrays using solid phase chemistry and photolabile protecting groups are disclosed, for example, in U.S. Pat. No. 5,744,305. These arrays can also be brought into contact with substances or substance libraries and tested for interaction, for example binding or changing conformation.

Thus, a substance to be tested can, for example, contain a detectable marker, for example, the substance can be radioactively labelled, fluorescence-labelled or luminescence-labelled. Furthermore, substances can be coupled to proteins which permit indirect detection, for example by means of enzymic catalysis employing a peroxidase assay which uses a chromogenic substrate or by means of binding a detectable antibody. Another possibility is that of investigating the array-bound protein complexes by means of mass spectrometry (SELDI). Changes in the conformation of a polypeptide which can be used in accordance with the invention as the result of interaction with a test substance can be detected, for example, by the change in the fluorescence of an endogenous tryptophan residue in the polypeptide. Thus, it has been possible to use SELDI to detect the interaction of the FGF-BP according to the invention with bFGF and subsequently to quantify this interaction in a radioactive ELISA (Tassi et al. (2001) J. Biol. Chem. 276:40247-53).

Pharmacologically active substances of the polypeptides which can be used in accordance with the invention can also be nucleic acids which are isolated using selection methods such as SELEX (see Jayasena, 1999, Clin. Chem. 45:1628-50; Klug and Famulok, 1994, M. Mol. Biol. Rep. 20:97-107; Toole et al. (1996) U.S. Pat. No. 5,582,981). In the SELEX method, those molecules (aptamers) which bind with high affinity to a polypeptide which can be used in accordance with the invention are typically isolated, by means of repeated amplification and selection, from a large pool of different single-stranded RNA molecules. Aptamers can also be synthesized and selected in their mirror-image form, for example as the L-ribonucleotide (Nolte et al. (1996) Nat. Biotechnol. 14:1116-9; Klussmann et al. (1996) Nat. Biotechnol. 14:1112-5). Forms which have been isolated in this way enjoy the advantage that they are not degraded by naturally occurring ribonucleases and therefore possess greater stability.

As a rule, oligonucleotides are rapidly degraded by endonucleases or exonucleases, in particular by DNases and RNases which are found in the cell. It is therefore advantageous to modify the nucleic acid in order to stabilize it against degradation, thereby ensuring that a high concentration of the nucleic acid is maintained in the cell over a long period of time (Beigelman et al. (1995) Nucleic Acids Res. 23:3989-94; Dudycz (1995) WO 95/11910; Macadam et al. (1998) WO 98/37240; Reese et al. (1997) WO 97/29116). Typically, such a stabilization can be obtained by introducing one or more internucleotide phosphorus groups or by introducing one or more non-phosphorus internucleotides.

Suitable modified internucleotides are compiled in Uhlmann and Peymann (1990 Chem. Rev. 90, 544) (see also Beigelman et al. (1995) Nucleic Acids Res. 23:3989-94; Dudycz (1995) WO 95/11910; Macadam et al. (1998) WO 98/37240; Reese et al. (1997) WO 97/29116). Modified internucleotide phosphate radicals and/or non-phosphorus bridges in a nucleic acid which can be employed in one of the uses according to the invention contain, for example, methyl phosphonate, phosphorothioate, phosphoramidate, phosphorodithioate and/or phosphate esters, whereas non-phosphorus internucleotide analogues contain, for example, siloxane bridges, carbonate bridges, carboxymethyl esters, acetamidate bridges and/or thioether bridges. It is also the intention that this modification should improve the durability of a pharmaceutical composition which can be employed in one of the uses according to the invention.

Within the context of the present invention, there is also the possibility of using antisense oligonucleotides (Zheng and Kemeny, 1995, Clin. Exp. Immunol. 100:380-2; Nellen and Lichtenstein, 1993, Trends Biochem. Sci. 18:419-23; Stein (1992) Leukemia 6:697-74) and/or ribozymes (Amarzguioui et al. (1998) Cell. Mol. Life. Sci. 54:1175-202; Vaish et al. (1998) Nucleic Acids Res. 26:5237-42; Persidis (1997) Nat. Biotechnol. 15:921-2; Couture and Stinchcomb (1996) Trends Genet. 12:510-5) to decrease the stability of nucleic acids and/or inhibit the translation of nucleic acids. Thus, by means of this use, according to the invention, of the nucleic acid sequences, it is possible, for example, to decrease the expression of the corresponding genes in cells both in vivo and in vitro. A single-stranded DNA or RNA is preferred for the use as a probe or as an antisense oligonucleotide.

The effects of modulating an FGF-BP which can be used in accordance with the invention or the identified, pharmacologically active substances which can be used in accordance with the invention can be investigated using various test systems which determine the activity of the cells and cell formations in vitro and in vivo.

Furthermore, the nucleic acids which encode the FGF-BP according to the invention can be contained in knock-out gene constructs or expression cassettes. Such knock-out gene constructs are known to the skilled person, for example, from U.S. Pat. Nos. 5,625,122; 5,698,765; 5,583,278 and 5,750,825.

Transgenic, non-human mammals, whose genome contains at least one knock-out gene construct which can be used in accordance with the invention and/or at least one expression cassette which can be used in accordance with the invention, as previously described, can be used for diagnosing and/or preventing and/or treating wound healing disturbances, in particular diabetes-associated wounds, or for identifying pharmacologically active substances. Depending on the promoter employed, transgenic animals which contain one of the previously described expression cassettes generally exhibit an expression of the nucleic acids and/or polypeptides which is elevated in a tissue-specific manner and can be used for analysing wound healing disturbances. Thus, an activin A-transgenic mouse, for example, exhibits improved wound healing (Munz et al. (1999) EMBO J. 18:5205-15) whereas a transgenic mouse possessing a dominantly negative KGF receptor exhibits delayed wound healing (Werner et al. (1994) Science 266:819-22). In addition to this, previously described transgenic animals can be provided with accelerated wound healing.

Methods for preparing transgenic animals, in particular transgenic mice, are likewise known to the skilled person from DE 196 25 049 and the U.S. Pat. Nos. 4,736,866; 5,625,122; 5,698,765; 5,583,278 and 5,750,825 and encompass transgenic animals which can be produced, for example, by the direct injection of expression vectors (see above) into embryos or spermatocytes, by the transfection of expression vectors into embryonic stem cells (Polites and Pinkert: DNA Microinjection and Transgenic Animal Production, pages 15 to 68 in Pinkert, 1994: Transgenic animal technology: a laboratory handbook, Academic Press, London, UK; Houdebine, 1997, Harwood Academic Publishers, Amsterdam, The Netherlands; Doetschman: Gene Transfer in Embryonic Stem Cells, pages 115 to 146 in Pinkert, 1994, see above; Wood: Retrovirus-Mediated Gene Transfer, pages 147 to 176 in Pinkert, 1994, see above; Monastersky: Gene Transfer Technology: Alternative Techniques and Applications, pages 177 to 220 in Pinkert, 1994, see above) or by isolating cell nuclei from differentiated somatic cells, for example fibroblasts, and subsequently transferring them into enucleated oocytes (McCreath et al. (2000) Nature 405:1004-5).

If the previously described nucleic acids which can be used in accordance with the invention are integrated into what are termed targeting vectors or knock-out gene constructs (Pinkert, 1994, see above), it is possible, after transfecting embryonic stem cells, and homologous recombination, to generate, for example, knock-out mice which, in general, as heterozygous mice, exhibit reduced expression of the nucleic acid, whereas homozygous mice no longer exhibit any expression of the nucleic acid. Animals which have been produced in this way can also be used for analysing wound healing disturbances. Thus, the eNOS (Lee et al. (1999) Am. J. Physiol. 277:H1600-H1608), Nf-1 (Atit et al. (1999) J. Invest. Dermatol. 112:835-42) and osteopontin (Liaw et al. (1998) J. Clin. Invest. 101:967-71) knock-out mice, for example, exhibit impaired wound healing. In this case, too, a tissue-specific reduction in the expression of wound healing-relevant genes, for example in skin-specific cells using the Cre-loxP system (stat3 knock-out, Sano et al. (1999) EMBO J. 18:4657-68), is particularly to be preferred. Cells or animals which are transgenic and knock-out for the FGF-BP protein according to the invention, and which have been produced in this way, can also be used, for example, for screening, for identifying pharmacologically active substances and/or for testing the modulation efficiency of vectors which are active in gene therapy.

The pharmacologically active substances which have been identified with the aid of the test methods which can be used in accordance with the invention can be employed, where appropriate combined or together with suitable additives and auxiliary substances, for preparing a diagnostic agent or medicament for diagnosing, preventing and/or treating wound healing disturbances which are characterized by a reduced quantity of FGF-BP, in particular in connection with diabetes-associated, poorly healing wounds.

The present invention furthermore relates to the use of at least one FGF-BP polypeptide which can be used in accordance with the invention, and which is as depicted in SEQ ID No. 1 and 3, or functional variants thereof, and/or FGF-BP polypeptide-encoding nucleic acids as depicted in SEQ ID No. 2 and 4, or a variant thereof, or antibodies or antibody fragments which are directed against an FGF-BP polypeptide which can be used in accordance with the invention, or functional variants thereof, and/or at least one cell which is expressing an FGF-BP polypeptide, where appropriate combined or together with suitable additives and auxiliary substances, for preparing a diagnostic agent for diagnosing wound healing disturbances which are characterized by a reduced quantity of FGF-BP, in particular diabetes-associated wounds.

For example, according to the present invention, one of the above-described nucleic acids can be used to prepare a diagnostic agent on the basis of the polymerase chain reaction (Examples 1 and 2, PCR diagnosis, e.g. in accordance with EP 0 200 362) or of an RNase protection assay (see, for example, Sambrook et al., above, Chapter 7, pages 7.71-7.78; Werner et al. (1992) Growth Factors and Receptors: A Practical Approach 175-197; Werner (1998) Proc. Natl. Acad. Sci. U.S.A. 89:6896-6900). These tests are based on the specific hybridization of a nucleic acid with its complementary counterstrand, usually the corresponding mRNA or its cDNA. In this connection, the nucleic acids which can be used in accordance with the invention can also be modified, as disclosed, for example, in EP 0 063 879. Preferably, such a DNA fragment is labelled by well-known methods using suitable reagents, for example radioactively using β-32P-dCTP or non-radioactively using biotin or digoxigenin, and incubated with isolated RNA which has preferably previously been bound to suitable membranes consisting, for example, of nitrocellulose or nylon. When the quantity of RNA which is analysed from each tissue sample is the same, it is then possible, in this way, to determine the quantity of mRNA which has been specifically labelled by the probe. Alternatively, the mRNA quantity can also be determined directly in tissue sections by means of in situ hybridization (see, for example, Werner et al. (1992) Proc. Natl. Acad. Sci. U.S.A. 89:6896-900).

A diagnostic agent which can be used in accordance with the invention can be employed to specifically measure, in vitro, the strength of the expression of the given gene in a tissue sample in order, for example, to be able to diagnose a wound healing disturbance reliably (Table 3). Such a method is particularly suitable for the early prognosis of disturbances and/or for ascertaining a predisposition for the development of a wound healing disturbance which is characterized by a reduced quantity of FGF-BP, in particular diabetes-associated wounds.

For example, a diagnostic agent which can be used in accordance with the invention for diagnosing skin diseases which are characterized by a reduced quantity of FGF-BP comprises the use of at least one nucleic acid encoding FGF-BP polypeptide or a functional variant thereof, as depicted in SEQ ID No. 2 and 4, and/or of functional variants of these nucleic acids, as a probe, preferably as a DNA probe and/or as a primer. Within the meaning of the present invention, a probe is a defined DNA or RNA fragment which is radioactively or chemically labelled and used for locating specific nucleic acid sequences by means of hybridization. Within the meaning of the present invention, a primer is a short DNA sequence which contains a free 3′ hydroxyl group and binds to a complementary strand. The primer is then the starting point for making a copy of the complementary strand. This opens up another possibility of obtaining the above-described nucleic acids, for example by using a suitable probe to isolate them from a suitable gene library, for example from a wound-specific gene library (see, for example, J. Sambrook et al. (1989) Molecular Cloning. A Laboratory Manual 2nd edition, Cold Spring Harbor Laboratory, Cold Spring Harbor, N.Y., Chapter 8, pages 8.1 to 8.81, Chapter 9, pages 9.47 to 9.58, and Chapter 10, pages 10.1 to 10.67).

Examples of suitable probes are DNA or RNA fragments which have a length of approx. 100-1000 nucleotides, preferably a length of approx. 200-500 nucleotides, in particular a length of approx. 300-400 nucleotides, whose sequence can be derived in accordance with SEQ ID No. 2 and SEQ ID No. 4 of the sequence listing and/or using the cDNA sequences of the database entries given in Table 1.

Alternatively, the derived nucleic acid sequences can be used for synthesizing oligonucleotides which are suitable for use as primers for a polymerase chain reaction. These primers can then be used to amplify and isolate the previously described nucleic acids, or parts thereof, from cDNA, for example wound-specific cDNA (Examples 1 and 2). Examples of suitable primers are DNA fragments which have a length of from approx. 10 to 100 nucleotides, preferably a length of from approx. 15 to 50 nucleotides, in particular a length of from 20 to 30 nucleotides, and whose sequence can be derived from SEQ ID No. 2 and SEQ ID No. 4 of the sequence listing and/or using the cDNA sequences of the database entries given in Table 1.

Another embodiment of the invention relates to the use of an antibody or antibody fragment, preferably of a polyclonal or monoclonal antibody or antibody fragment, for the analysis and diagnosis of wound healing disturbances which are characterized by a reduced quantity of FGF-BP and its use for identifying pharmacologically active substances, characterized in that an antibody-producing organism is immunized with a polypeptide which can be used in accordance with the invention.

These antibodies can be used, for example, for readily and rapidly examining a tissue sample so as to determine whether the polypeptide in question is present in a reduced quantity in order, thereby, to obtain an indication of a possible disease, in particular of skin diseases which are characterized by a reduced quantity of FGF-BP, in particular diabetes-associated wounds. In this case, the antibodies according to the invention are, for example, labelled with an enzyme, as has already been described above. As a result of this, the specific antibody/peptide complex can readily, and just as rapidly, be detected by means of an enzymic colour reaction.

It is also possible, for example, for the local injection of monoclonal antibodies directed against TGF beta 1 to improve wound healing in an animal model (Ernst et al. (1996) Gut 39:172-5).

The procedure for preparing an antibody or antibody fragment, preferably a polyclonal or monoclonal antibody, is effected, in accordance with methods which are well known to the skilled person, by immunizing a mammal, for example a rabbit, with the polypeptide according to the invention, or functional variants thereof, preferably with parts thereof having a length of at least 6 amino acids, preferably having a length of at least 8 amino acids, in particular having a length of at least 12 amino acids, where appropriate in the presence of, for example, Freund's adjuvant and/or aluminium hydroxide gels (see, for example, Diamond, B. A. et al. (1981) The New England Journal of Medicine: 1344-1349). The polyclonal antibodies which are formed in the animal as a result of an immunological reaction can subsequently be readily isolated from the blood using well known methods and, for example, purified by means of column chromatography. Monoclonal antibodies can, for example, be prepared in accordance with the known method of Winter & Milstein (Winter, G. & Milstein, C. (1991) Nature, 349, 293-299). As an alternative to the classical antibodies, it is also possible, for example, to use what are termed “anticalins”, which are based on lipocalin (Beste et al. (1999) Proc. Natl. Acad. Sci. USA, 96: 1898-1903). The natural ligand-binding sites of the lipocalins, for example the retinol-binding protein or the bilin-binding protein, can be altered, for example by means of a “combinatorial protein design” approach, in such a way that they bind to selected haptens, for example to the polypeptide which can be used in accordance with the invention (Skerra, 2000, Biochim. Biophys. Acta 1482:337-50). Other known “scaffolds” are known as being alternatives to antibodies for molecular recognition (Skerra, J. Mol. Recognit., 2000, 13:167-187).

The antibody which can be used in accordance with the invention, or the antibody fragment, are directed against a polypeptide according to the invention and react specifically with the polypeptides according to the invention, with the abovementioned parts of the polypeptide either themselves being immunogenic, or with it being possible to make them immunogenic, or for their immunogenecity to be increased, by coupling them to suitable carriers, such as bovine serum albumin. This antibody which can be used in accordance with the invention is either polyclonal or monoclonal, with a monoclonal antibody being preferred. According to the present invention, the term antibody or antibody fragment is also understood as meaning antibodies, or antigen-binding parts thereof, which have been prepared recombinantly and, where appropriate, modified, such as chimaeric antibodies, humanized antibodies, multifunctional antibodies, bi- or oligospecific antibodies, single-stranded antibodies and F(ab) or F(ab)2 fragments (see, for example, EP-B1-0 368 684, U.S. Pat. No. 4,816,567, U.S. Pat. No. 4,816,397, WO 88/01649, WO 93/06213, WO 98/24884). Thus, antibodies directed against the FGF-BP according to the invention have already been successfully prepared and used (Liu et al. (2001) Kidney Int., 59:1717-1728; Sauter et al. (2001) Int. J. Cancer 92:374-381).

In order to use pharmacologically active substances as diagnostic agents, it is possible for the substances to contain a detectable label; for example, the substance can be radioactively labelled, fluorescence-labelled or luminescence-labelled. Furthermore, substances can be coupled to enzymes which permit indirect detection, for example, as described above, by way of enzymic catalysis, by means of a peroxidase assay using a chromogenic substrate, or by binding a labelled or detectable antibody. The substances can then be brought into contact with a sample which is to be investigated and, in this way, the quantity of a polypeptide which can be used in accordance with the invention, or of a functional variant thereof, or of a nucleic acid encoding this polypeptide or variant, or of a variant thereof, or of a cell which is expressing a polypeptide which can be used in accordance with the invention, or a functional variant thereof, or a nucleic acid encoding this polypeptide or variant, or a variant thereof, or of an antibody or an antibody fragment which is directed against a polypeptide which can be used in accordance with the invention, in the sample can be determined. The results obtained with this sample, which is derived from an organism to be investigated, can then be compared with the result obtained from a sample which is derived from a healthy or diseased organism.

The invention also relates to the use of at least one polypeptide which can be used in accordance with the invention, or a functional variant thereof, or a nucleic acid encoding this polypeptide or variant, or a variant thereof, or an antibody or an antibody fragment which is directed against a polypeptide which can be used in accordance with the invention, or a functional variant thereof, where appropriate combined or together with suitable additives and auxiliary substances, for preparing an array, which is fixed on a support material, for performing analyses in connection with wound healing disturbances which are characterized by a deficiency of FGF-BP, in particular diabetes-associated, poorly healing wounds.

Methods for preparing such arrays using solid phase chemistry and photolabile protecting groups are disclosed, for example, in U.S. Pat. No. 5,744,305.

For performing the analyses in connection with wound healing disturbances which are characterized by a reduced quantity of FGF-BP, in particular diabetes-associated wounds, it is also possible, for example, to use DNA chips and/or protein chips which comprise at least one nucleic acid, at least one polypeptide and/or at least one antibody or antibody fragment as previously described. DNA chips are disclosed, for example, in U.S. Pat. No. 5,837,832.

The invention also relates to the use of at least one FGF-BP polypeptide as depicted in SEQ ID No. 1 or SEQ ID No. 3, or functional variants thereof, and/or of at least one FGF-BP encoding nucleic acid as depicted in SEQ ID No. 2 or SEQ ID No. 4, or functional variants thereof and/or of an antibody or of antibody fragments directed against an FGF-BP polypeptide or functional variants thereof, and/or of a cell which is expressing an FGF-BP polypeptide, or functional variants thereof, for preparing a medicament for treating and/or preventing diabetes-associated wound healing disturbances or for diagnosing diabetes-associated wound-healing disturbances.

The FGF-BP encoding nucleic acid or functional variants thereof can be used in this context in the form of an expression vector, in particular of a vector which is effective in gene therapy. In case of using FGF-BP polypeptide or of a functional variant thereof, it is preferably a recombinant polypeptide, that can be isolated ideally from bacteria like E. coli, from yeasts like S. cerevisiae or from Schizosaccharomyces pombe or from viruses, in particular Baculoviruses.

The FGF-BP polypeptide, the FGF-BP encoding nucleic acid, the antibody or antibody fragments directed against FGF-BP, used as a drug or therapeutic/medicament are administered systemically or locally, in particular locally introduced into the wound and/or applying topically. The FGF-BP polypeptide, the FGF-BP encoding nucleic acid, the antibody directed against the FGF-polypeptide or antibody fragments as well as variants and derivatives thereof can also be administered prophylactically to the skin of diabetes patients not affected by a wound, for example in form of an appropriate ointment, lotion, oil or cream for preventing diabetes-associated wounds.

The diabetes associated wound is particularly preferably a wound which heals poorly, in particular a diabetic ulcer.

The present invention relates also to a method for treating wound healing disturbances, in particular diabetes-associated wound healing disturbances which are not necessarily characterized by reduced quantity of FGF-BP. The method encompasses the step (i) the administration of a therapeutically active quantity of FGF-BP polypeptide, of an FGF-BP encoding nucleic acid sequence and/or of an antibody or antibody fragment directed against an FGF-BP to a patient.

If necessary, before or after administration, the quantity of FGF-BP in a wound or at the edge of the wound can be determined.

All above described preferred embodiments can be applied to the method.

The invention will now be further clarified below with the aid of the tables and examples without the invention being restricted thereto.

### Tables

Table 1 lists the FGF-BP polypeptides and nucleic acids which can be used in accordance with the invention.
Table 2 shows the kinetics, determined by “Taq-Man” analysis, of the differential regulation of FGF-BP expression in the wound healing seen in normal mice and in diabetic mice, as an example of a wound healing disturbance which is connected with a deficiency of FGF-BP.
Table 3 shows the differential regulation, determined by “Taq-Man” analysis, of FGF-BP expression as compared in three different wound healing states in humans (normal wound healing and venous and diabetic ulcer), as an example of the predisposition, which occurs selectively in diabetic patients, for a wound healing disturbance which is caused by a deficiency of FGF-BP.
Table 4 shows the results of disruption tests which are carried out on wounds treated with FGF-BP expressing plasmid on diabetic rats.

The polypeptide sequences of murine and human FGF-BP, and also their encoding nucleotide sequences, are listed in accordance with SEQ ID No. 1 to SEQ ID No. 4.

The oligonucleotides which are used for the examples are listed in accordance with SEQ ID No. 5 to SEQ ID No. 12.

### EXAMPLES

### Example 1

### Differential Expression of FGF-BP mRNA in Human Biopsies Taken from the Healthy Skin and Wounds of Healthy Test Subjects and in Biopsies Taken from Venous and Diabetic Ulcers

In a first experiment, the intention was to investigate the extent to which the expression of FGF-BP is regulated in various wound healing diseases. For this, 4 mm or 6 mm punches were used to remove skin samples from the untreated intact skin, from the wounds at 1 hour after wounding, from the day 1 wound and from the day 5 and day 15 wounds, of 6 healthy test subjects, and the biopsies taken at each time point were pooled. In addition, punch biopsies of intact skin and also of the bottom and edge of the wound were taken, at the same time, from 6 patients suffering from chronic venous ulcers and from 4 patients suffering from diabetic ulcers and each group of biopsies (intact skin, wound bottom and wound edge) were subsequently pooled separately.

RNA was isolated from all the biopsies. For this, a disperser was used to homogenize the biopsies in RNAclean buffer (AGS, Heidelberg) to which 1/100th volume of 2-mercaptoethanol had been added. The RNA was subsequently extracted by phenolizing twice using water-saturated, acidic phenol in the presence of 1-bromo-3-chloropropane. An isopropanol precipitation and an ethanol precipitation were subsequently carried out and the RNA was washed with 75% ethanol. After that, the RNA was digested with DNase I. For this, 20 μg of RNA (made up to 50 μl with DEPC-treated water) were incubated, at 37° C. for 20 min, with 5.7 μl of transcription buffer (Roche), 1 μl of RNase inhibitor (Roche; 40 U/μl) and 1 μl of DNase I (Roche; 10 U/μl). 1 μl of DNase I was then added once again and the mixture was incubated at 37° C. for a further 20 min. The RNA was subsequently phenolized, precipitated with ethanol and washed. All the above-listed steps were carried out using DEPC (diethyl pyrocarbonate)-treated solution or liquids provided these latter did not contain any reactive amino groups.

cDNA was subsequently prepared from the extracted RNA. This took place in the presence of 1×Taq Man RT buffer (Perkin Elmer), 5.5 mM MgCl2 (Perkin Elmer), in each case 500 μM dNTPs (Perkin Elmer), 2.5 μM random hexamers (Perkin Elmer), 1.25 U of MultiScribe reverse transcriptase/μl (50 U/μl, Perkin Elmer), 0.4 U of RNase inhibitor/μl (20 U/μl, Perkin Elmer), 20 μl of RNA (50 ng/μl) and DEPC-treated water (up to a volume of 100 μl). After the RNA had been added and after the solution had been mixed thoroughly, it was aliquoted in two 0.2 ml tubes (50 μl in each case) and the reverse transcription was carried out in a temperature cycler (10 min at 25° C.; 30 min at 48° C. and 5 min at 95° C.). The cDNA was subsequently quantified by means of quantitative PCR using the SYBR Green PCR master mix (Perkin Elmer), with the quantity of FGF-BP cDNA being determined in triplicate. For this, the FGF-BP-encoding SEQ ID No. 1 was used to select the primers as depicted in SEQ ID No. 5 and SEQ ID. No. 6. Cyclophilin A (Gen Bank: XM039526), which was amplified using the primers having the sequences as depicted under SEQ ID No. 7 and SEQ ID No. 8, was used as the reference. With a total volume of 57 μl, the stock solution for each triplet contained 37.5 μl of 2×SYBR Master Mix, 0.75 μl of AmpErase UNG (1 U/μl) and 18.75 μl of DEPC-treated water. For each determination in triplicate, in each case 1.5 μl of forwards and backwards primer (as depicted in SEQ ID No. 5 and SEQ ID No. 6) were added, in a previously optimized concentration ratio, to 57 μl of stock solution. In each case 60 μl of the stock solution/primer mixture were mixed with 15 μl of cDNA solution (2 ng/μl) and the whole was aliquoted into three wells. In parallel to this, a stock solution containing primers for determining cyclophilin A (SEQ ID No. 7 and SEQ ID No. 8) was prepared and then mixed with a further 15 μl of the same cDNA solution, after which the whole was aliquoted into three wells.

In addition, different cDNA solutions were prepared as a dilution series (4 ng/μl; 2 ng/μl; 1 ng/μl; 0.5 ng/μl and 0.25 ng/μl) for the purpose of constructing a standard curve for the cyclophilin PCR. In each case, 15 μl of these cDNA solutions were mixed with 60 μl of stock solution/primer mixture for determining cyclophilin and aliquoted into three wells. A standard curve for the human FGF-BP PCR was also constructed; when doing this, the same dilutions were used as those which were also employed for the cyclophilin standard curve. A PCR mixture without cDNA served as the control. In each case 15 μl of DEPC-water were added to 60 μl of stock solution/primer mixture for human FGF-BP, on the one hand, and cyclophilin, on the other hand, after which each separate 75 μl volume was mixed and in each case aliquoted into three wells. The mixtures were amplified in a GeneAmp 5700 (2 min at 50° C.; 10 min at 95° C., followed by 3 cycles of 15 sec at 96° C. and 2 min at 60° C.; after that 37 cycles of 15 sec at 95° C. and 1 min at 60° C.). The analysis was effected by determining the relative abundance of FGF-BP in relation to the cyclophilin reference. For this, a standard curve was first of all constructed by plotting the CT values of the dilution series against the logarithm of the quantity of cDNA in the PCR mixture (in ng of transcribed RNA), and the slope(s) of the straight lines were determined. The efficiency (E) of the PCR is then given by the following equation: E=10−1/s−1. The relative abundance (X) of the FGF-BP cDNA species investigated (Y) in relation to cyclophilin is then:

X=(1+Ecyclophilin)CT(cyclophilin)/(1+EY)CT(Y). The numerical values were subsequently standardized by making the quantity of cDNA obtained from the intact skin of healthy test subjects equal to 1.

The relative changes in the expression of FGF-BP in various wound healing states are compiled in Table 3. It is found that a 2-fold to 3-fold increase in the expression of FGF-BP, as compared with that in intact skin, can be observed in all wound healing states in healthy test subjects while patients who are suffering from venous, poorly healing ulcers always exhibit an elevated expression of FGF-BP. By contrast, patients who are suffering from diabetic, poorly healing ulcers always express too little FGF-BP. It follows from this that the cause of the poor wound healing in diabetic patients results from a general, but selective for this disease as compared, for example, with venous wounds, decrease in the abundance of FGF-BP. Consequently, as compared with healthy test subjects and patients who suffer from venous ulcers, diabetic patients are predisposed to poorer wound healing due to a deficiency in FGF-BP. The results of this experiment demonstrate that a promising therapy for wound healing disturbance, in particular diabetes-associated wounds, may consist in locally increasing the quantity and/or activity of FGF-BP polypeptides and/or nucleic acids and consequently achieving improved wound healing. The results further demonstrate that the increased FGF-BP expression in ulcer patients as compared with intact, normal skin from healthy patients is clearly below the expression values in wounds from healthy patients, especially at the edge of the wound. It follows from this that patients, in particular patients suffering from ulcus, who do not per se exhibit a reduced expression of FGF-BP, can be treated with FGF-BP.

### Example 2

### Differential Expression of FGF-BP in Murine Wounds

In order to confirm that FGF-BP is in fact particularly suitable for treating, diagnosing and/or preventing wound healing disturbances which are characterized by a deficiency in FGF-BP, in particular diabetes-associated, poorly healing wounds, the influence of FGF-BP on wound healing was investigated in animal models. Additionally, diabetes-associated wounds that are not necessarily characterized by a deficiency in FGF-BP are of particular general interest. Confirmation of the human data in animal models is a prerequisite for determining suitable time points for treating, diagnosing and/or preventing wound healing disturbances which are characterized by a deficiency in FGF-BP, in particular diabetes-associated wounds. This applies in general to all diabetes-associated wounds, also to wounds which are not characterized by a reduced quantity of FGF. Since mice have proved to be a suitable model system for investigating wound healing in humans, the expression of FGF-BP was determined in murine biopsies taken at different times during wound healing. In this connection, punch biopsies were taken from the wounds, at 1 h, 7 h, 15 h, 24 h, 3 days, 5 days, 7 days and 14 days after wounding, and from the intact skin, of normally healing control mice (C57BL/6) and diabetic mice (C57BL/Ks-db/db/Ola).

The biopsies were homogenized, and RNA isolated, as described in Example 1. After that, the RNA was digested with DNase and transcribed into cDNA. Wound healing-relevant cDNAs were likewise quantified as described in Example 1. Primers having the sequences of SEQ ID No. 9 and SEQ ID No. 10 were selected, with the aid of the sequence for FGF-BP (SEQ ID No. 4), for specifically amplifying murine FGF-BP. In this experiment, GAPDH was used as the reference gene. For this PCR amplification, use was made of primers having the sequences of SEQ ID No. 11 and SEQ ID No. 12, these primers being determined with the aid of the sequence of the GAPDH gene (Gen Bank: M17851). For the quantification, cDNA from 10 ng of reverse-transcribed total RNA was amplified, per assay, in a total volume of 25 μl. The PCR was carried out in accordance with the instructions of the manufacturer (PE Applied Biosystems, SYBR Green PCR and RT-PCR Reagents Protocol, 1998). The CT values were analysed and the frequency of FGF-BP mRNA relative to that of GAPDH mRNA was calculated from these values. The results of the experiments are compiled in Table 2 and demonstrate that in diabetic animals, during the whole of the observation period, FGF-BP mRNA is expressed during wound healing to an extent which is markedly reduced (on average by a factor of 2.8) as compared with the extent of its expression in intact skin. When these results are compared with the data for wound healing kinetics in normal mice, it is found that the relative expression of FGF-BP mRNA remains virtually unaltered during wound healing in normal mice. These results make it clear that the selective decrease in the expression of FGF-BP which is observed in human diabetic patients, and which is taken to be a cause of the wound healing disturbances in this patient group, can also be applied to the mouse. Consequently, the diabetic mouse can be used as a model for establishing data relative to therapy, such as suitable administration times and forms.

### Example 3a

### Treating Murine Wounds with FGF-BP Using Gene Therapy Methods

In order to confirm that FGF-BP is in fact suitable particularly for treating and/or preventing diabetes-associated, poorly healing wounds, the influence of FGF-BP on wound healing is tested in vivo. For this, wound healing is monitored in male diabetic Sprague Dawley rats following administration of the FGF-BP cDNA as depicted in SEQ ID No. 4. For quantifying the wound healing, the tensile strength of the wounds is investigated, with a higher tensile strength reflecting improved wound healing. The diabetic rat animal model is an established model system when investigating diabetes-associated wounds which heal poorly (Davidson, Arch. Dermatol. Res. (1998) 290: pp. 1-11).

An expression vector which is suitable for this experiment is first of all constructed, for example on the basis of the vector pMH (F. Hoffman-La Roche), by inserting intron II of the rat insulin gene into the HindIII cleavage site, between the CMV promoter and the multiple cloning site (to give pMHInt). The FGF-BP cDNA according to the invention is then cloned into the modified expression vector pMHInt using the multiple cloning site. For this, the coding region of the FGF-BP cDNA as depicted in SEQ ID No. 4 is amplified by means of a suitable PCR and subsequently cut with restriction enzymes which permit the cDNA to be ligated in-frame into the modified expression vector, which has been cut with the same restriction enzymes. This results in an expression plasmid, i.e. pMHInt_FGF-BP. pMHInt containing, for example, a luciferase gene (pMHIntLuc), is used as the control vector.

In order to induce the diabetes, four rats each having a body weight of 250-300 g are injected i.p. with a freshly prepared aqueous solution of Streptozotocin (Sigma) (50 mg/kg of body weight). The blood sugar of the animal is tested 7-9 days after the induction. A value for the blood sugar level of more than 200 mg/dL is taken as the lower limit when assessing the diabetic state of the animals.

The four diabetic rats, and also the four non-diabetic control animals, are subsequently anaesthetized with a mixture of 2% of O2 (2 l/min) and 1.25% isoflurane. The back of each animal is depilated and marks are made at four sites on the back of each animal for subsequent wounding. A Helios gene gun (BioRad) is used to bombard each of these marks, at 500 psi, with, for example, in each case 0.5 μg of plasmid DNA which is bound to gold particles (BioRad), with in each case two sites being bombarded with the FGF-BP expression vector pMHInt_FGF-BP and two sites being bombarded with the control vector pMHIntLuc. In this connection, any bombardment with pMHInt_FGF-BP in each case takes place at the anterior end of the mark and at the posterior end of the mark. Subsequently, incision wounds of 1 cm in length are made through the bombarded sites and the wounds are closed with wound clips. After 10 days, the tensile strength of the wounds is determined using a tensiometer (BTC-2000, SRLI, Nashville) in accordance with the manufacturer's instructions. Subsequently, the quotient (E/C value) is generated from the absolute value of the tensile strength of a wound bombarded with pMHInt_FGF-BP and the absolute value of the tensile strength of a wound in the same animal which has been bombarded with the control vector pMHIntLuc. Determining the means of the E/C values makes it possible to determine the changes, in diabetic and control animals, which occur in the tensile strength in dependence on the presence of FGF-BP according to the invention. In this connection, an increase in the tensile strength of pMHInt_FGF-BP-treated wounds in diabetic animals points to FGF-BP being suitable for selectively treating diabetes-associated, poorly healing wounds.

Alternatively, it is naturally also possible to select other expression vectors and other ways of administering the nucleic acids. Thus, nucleic acids can, for example, be complexed with liposomes in order to facilitate penetration into the cell, or the nucleic acid can be bound to a support matrix, for example collagen, with this matrix then being applied to the wound. This enables the nucleic acid to penetrate into the skin cells. In order to overcome the difficulty in penetrating into the cell, it is also possible to use viral vectors, for example adenoviruses or retroviruses, which then obtain access to the cells by means of infection.

It is also possible, for example, to use human FGF-BP polypeptide-encoding nucleic acids as depicted in SEQ ID No. 2 or variants thereof.

### Example 3b

### Treating Wounds with FGF-BP by Means of Gene Therapy Based on the General Scheme of Example 3a

The expression vector pMHIint_FGF-BP has been prepared on the basis of the vector pMH, as described in example 3a). As a control vector, pMHInt was used. In order to induce diabetes, rats having a body weight of 250-300 g were injected i.p. with a freshly prepared aqueous solution of Streptozotocin (Sigma) (50 mg/kg of body weight). The blood sugar of the animals was tested 5, 10 and 17 days after the induction. A value for the blood sugar level of more than 200 mg/dL is taken as the lower limit when assessing the diabetic state of the animals. At day 10 after the induction of the diabetic state the wounds were made. For this, 28 diabetic rats were anaesthetized with a mixture of 2% O₂, (2 l/min) and 1.25% of isoflurane. The back was depilated and marks were made at four sites of the back of each animal for subsequent wounding. A Helios gene gun (BioRad) was used to bombard each of these marks, at 500 psi, with in each case 0.5 μg of plasmid DNA bound to gold particles (BioRad), with 16 animals being bombarded with the FGF-BP expression vector pMHInt_FGF-BP and 12 animals being bombarded with the control vector pMHInt. Subsequently, incision wounds of 1.9 cm in length were made through the bombarded sites and the wounds were closed with wound clips. After 7 days, the tensile strength of the wounds was determined using a tensiometer (BTC-2000, SRLI; Nashville) in accordance with the manufacturere's instructions. The more negative the pressure (in [mmHg]) that can be applied to a wound before it ruptures, the higher the tensile strength of the wound. The results of the experiment are shown, in table 4. Due to the more simple presentation, all negative pressure values in the result of the experiment are indicated as positive values, with a higher value indicating a better tensile strength of the wound. In the table, single values are indicated. The lower number of values results from the non-evaluable state of some wounds.

It has been observed that the wounds of animals which had been bombarded with the FGF-BP expression vector pMHInt_FGF-BP showed a higher tensile strength compared to wounds having been bombarded with the control vector.

This proves a particular suitability of FGF-BP for treating diabetes-associated poorly healing wounds.

### Example 4

### Treating Murine Wounds by the In Vivo Administration of FGF-BP Polypeptides

Since the FGF-BP polypeptide which can be used in accordance with the invention has an extracellular site of action, and penetration into the cells is consequently not required, the suitability of topically administered, recombinantly prepared FGF-BP polypeptides for treating and/or preventing diabetes-associated, poorly healing wounds is tested in vivo.

First of all, FGF-BP polypeptides are prepared, for example, using the baculovirus expression system (BAC-TO-BAC Baculovirus Expression System, Life Technologies Inc., Gaithersburg, Md.). For this, the cDNA for the FGF-BP which can be used in accordance with the invention, as depicted in SEQ ID No. 4, is first of all amplified by means of PCR (see Experiment 3) and provided with a (His)6 tag at each end. This modified cDNA is cloned into suitable restriction cleavage sites in the pFASTBAC HTb donor plasmid. The plasmid is then transformed into the Bacmid-containing E. coli strain DH10BAC. Isolated, recombinant bacmid DNA is then transfected into Sf-9 cells in order to generate corresponding baculoviruses. After five days, the cells are harvested, pelleted and lysed using 6 M guanidinium HCl, 0.01 M HCl, 0.1 M sodium phosphate, pH 8.0. The cell lysates are incubated on ice for one hour, after which cell residues are centrifuged off and the supernatant is loaded onto a nickel-NTA Sepharose column (Qiagen, Hilden, Germany). The column is washed repeatedly with 30 mM sodium citrate, 300 mM sodium chloride and descending pH values of 8, 6.3, 5.9 and 5.7. His-tagged FGF-BP is then eluted with 3×0.5 ml of buffer having a pH of 4.5 and neutralized. Recombinant FGF-BP polypeptide which has been prepared in this way is then formulated at various concentrations, for example in an inert hydrogel (2.3% modified methyl cellulose, 20% propylene glycol and 77.7% water) (Intra Site from Smith & Nephew).

In order to induce diabetes, four rats are treated with streptozotocin solution as described in Experiment 3. Four of these diabetic rats, and also four non-diabetic control animals, are subsequently anaesthetized and depilated as already described. A biopsy punch is then used to punch four wounds in the depilated back skin of an animal. The formulated FGF-BP polypeptides are then applied, in a total volume of 50 μl per wound, to two of these wounds, while the carrier substance alone, without any FGF-BP, is applied topically to the edges of the remaining two wounds as a control. The wounds are dressed individually with a semiocclusive dressing (OpSite). The progression of the reepithelialization of the wound is documented photographically (EPICAM) for 10 days and compared with the value at day 0 (immediately after wounding). A rate of reepithelialization which is accelerated selectively in diabetic animals following the administration of FGF-BP polypeptides indicates that the FGF-BP polypeptides are particularly suitable for selectively treating diabetes-associated, poorly healing wounds.

Alternatively, it is also possible to use other expression systems which make it possible to prepare a correctly folded, biologically active FGF-BP polypeptide, and also other formulations, or else the intradermal injection of proteins in suitable excipient solutions. In addition to the murine protein, it is also possible to use human FGF-BP polypeptides as depicted in SEQ ID No. 1 or functional variants of these polypeptides.

In this way, it can be demonstrated that FGF-BP is active in preventing and/or treating diabetes-associated, poorly healing wounds. The effect of FGF-BP is particularly suitable for these diabetes-associated wound healing disturbances as compared with treating other wound healing disturbances since, in the latter case, there is no faulty regulation of FGF-BP expression.

For prevention and/or treatment, the quantity of FGF-BP, preferably of functionally active FGF-BP, should be increased in the region of the wound. An indication which is to be treated preferentially is the diabetic ulcer. A medicament comprising FGF-BP is preferably administered topically, in particular by means of gene therapy (Example 3). The administration can furthermore particularly preferably take place by means of the topical application of a recombinant FGF-BP polypeptide according to the invention (Example 4), since the site of action of the FGF-BP polypeptide is extracellular and penetration of the protein into cells is consequently not required.

TABLE 1

| | | | SEQ ID | | SEQ ID |
|---|---|---|---|---|---|
| Name | Organism | Protein | No. | cDNA | No. |
| Human | Homo | TrEMBL: | 1 | GenBank: | 2 |
| FGF-BP | sapiens | Q14512 | | M60047 | |
| Murine | Mus | TrEMBL: | 3 | GenBank: | 4 |
| FGF-BP | musculus | O70514 | | AF065441 | |

TABLE 2

| | Rel. quantity of mFGF- | Rel. quantity of mFGF- |
|---|---|---|
| Time after | BP cDNA in healthy | BP cDNA in diabetic |
| wounding | mice | mice |
| Intact skin | 1.0 | 1.0 |
| 1 d | 1.0 | 0.3 |
| 3 d | 1.0 | 0.5 |
| 5 d | 0.9 | 0.5 |
| 7 d | 1.1 | 0.3 |
| 14 d | 1.5 | 0.4 |

TABLE 3

| | Rel. quantity of |
|---|---|
| Biopsy | FGF-BP cDNA |
| Patient pool 1: | 1.00 |
| Intact skin | |
| Healthy patients | |
| Patient pool 2: | 3.10 |
| 1 hour wound | |
| Healthy patients | |
| Patient pool 3: | 2.70 |
| 1 day wound | |
| Healthy patients | |
| Patient pool 4: | 1.80 |
| 5 day wound | |
| Healthy patients | |
| Patient pool 5: | 1.10 |
| 15 day wound | |
| Healthy patients | |
| Patient pool 6: | 1.60 |
| Intact skin | |
| Ulcer patients | |
| Patient pool 7: | 1.50 |
| Edge of wound | |
| Ulcer patients | |
| Patient pool 8: | 0.01 |
| Bottom of wound | |
| Ulcer patients | |
| Patient pool 9: | 0.50 |
| Intact skin | |
| Patients having a diabetic ulcer | |
| Patient pool 10: | 0.60 |
| Edge of wound | |
| Patients having a diabetic ulcer | |
| Patient pool 11: | 0.00 |
| Bottom of wound | |
| Patients having a diabetic ulcer | |

TABLE 4

| pMHInt FGF-BP | pMHInt |
|---|---|
| (experiment - expression plasmid) | (control - vector only) |
| Values in mmHg, indicated positively | Values in mmHg, indicated positively |
| 386; 227; 318; 322; 274; 203; 621; 240; | 473; 114; 387; 494; 246; 322; 367; 409; |
| 189; 391; 401; 570; 504; 317; 321; 391; | 303; 518; 486; 340; 375; 449; 286; 253; |
| 329; 301; 603; 365; 263; 221; 261; 460; | 216; 261; 218; 350; 464; 389; 342; 409; |
| 279; 542; 641; 419; 255; 372; 401; 669; | 341; 334; 261; 304; 304; 286; 345; 498; |
| 328: 368; 401; 229; 277; 345; 390; 289; | 365; 293; 418; 250; 133; 397; 471; 463; |
| 377; 387; 297; 412; 354; 388; 296; 505; | 263; 253; 224; 252; 413; 302; 263 |
| 353; 372; 262; 310; 428; 384; 331; 383; | |
| 350; 422; 191; 318; 357 | |
| mean value: 363 | mean value: 338 |

1. Use of at least one FGF-BP polypeptide as depicted in SEQ ID No. 1 and SEQ ID No. 3, or functional variants thereof, and/or of at least one FGF-BP-encoding nucleic acid as depicted in SEQ ID No. 2 and SEQ ID No. 4, or functional variants thereof, and/or of antibodies or antibody fragments which are directed against an FGF-BP polypeptide which can be used in accordance with the invention, or functional variants thereof, and/or of a cell which is expressing an FGF-BP polypeptide, or functional variants thereof, for diagnosing wound healing disturbances which are characterized by a reduced quantity of FGF-BP.

2. The use according to claim 1, characterized in that the nucleic acid encoding FGF-BP, or functional variants thereof, is employed in the form of an expression vector, in particular of a vector which is active in gene therapy.

3. The use according to claim 1 or 2, characterized in that the FGF-BP polypeptide is a recombinant protein, preferably from bacteria, yeasts or viruses, in particular baculoviruses.

4. The use according to one of the claims 1 to 3, characterized in that the diabetes-associated wound is a poorly healing wound, in particular a diabetic ulcer.

5. The use of at least one FGF-BP polypeptide as depicted in SEQ ID No. 1 and SEQ ID No. 3, or functional variants thereof, or of at least one FGF-BP-encoding nucleic acid as depicted in SEQ ID No. 2 and SEQ ID No. 4, or functional variants thereof, or of antibodies or antibody fragments which are directed against an FGF-BP polypeptide, or functional variants thereof, and/or of one cell which is expressing an FGF-BP polypeptide or functional variants thereof, where appropriate together or combined with suitable additives and/or auxiliary substances, for diagnosing wound healing disturbances which are characterized by a reduced quantity of FGF-BP.

6. The use according to claim 5, characterized in that the said cells are human, non-embryonic, autologous or allogenic cells.

7. The use according to claim 5, characterized in that the cells are skin cells, in particular keratinocytes, fibroblasts or endothelial cells.

8. The use according to claim 5, characterized in that the nucleic acid is a DNA or RNA, preferably a DNA, particularly preferably a double-stranded DNA.

9. The use according to at least one of the preceeding claims, characterized in that the variant of SEQ ID No. 1 and/or 3 and, respectively, SEQ ID No. 2 and/or 4 is a fusion protein or, respectively, a fusion protein-encoding nucleic acid.

10. The use of at least one FGF-BP polypeptide as depicted in SEQ ID No. 1 and 3 and/or of FGF-BP polypeptide-encoding nucleic acids as depicted in SEQ ID No. 2 and 4 and/or of antibodies or antibody fragments which are directed against an FGF-BP polypeptide or functional variants thereof, and/or a cell expressing at least FGF-BP for preparing a diagnostic agent for diagnosing wound healing disturbances which are characterized by a reduced quantity of FGF-BP.

11. The use according to claim 10, characterized in that at least one nucleic acid as depicted in SEQ ID No. 2 and 4, which encodes an FGF-BP polypeptide or a functional variant thereof, and/or functional variants of these nucleic acids, is/are employed as a probe, preferably as a DNA probe, and/or as a primer, for diagnosing skin diseases which are characterized by a reduced quantity of FGF-BP.

12. The use according to claim 10, characterized in that an antibody or antibody fragments which is/are directed against an FGF-BP polypeptide or functional variants thereof are used.

13. The use according to at least one of claims 5-12, characterized in that the wound healing disturbance is a diabetes-associated wound, in particular a diabetic ulcer.


SEQUENCE LISTING (12 sequences)

SEQ ID NO: 1 - protein, 234 aa, Homo sapiens [sequence omitted]

SEQ ID NO: 2 - DNA, 705 bp, Homo sapiens
atgaagatctgtagcctcaccctgctctccttcctcctactggctgctcaggtgctcctggtggaggggaaaaaaaaagtgaagaatggacttcacagcaaagtggtctcagaacaaaaggacactctgggcaacacccagattaagcagaaaagcaggcccgggaacaaaggcaagtttgtcaccaaagaccaagccaactgcagatgggctgctactgagcaggaggagggcatctctctcaaggttgagtgcactcaattggaccatgaattttcctgtgtctttgctggcaatccaacctcatgcctaaagctcaaggatgagagagtctattggaaacaagttgcccggaatctgcgctcacagaaagacatctgtagatattccaagacagctgtgaaaaccagagtgtgcagaaaggattttccagaatccagtcttaagctagtcagctccactctatttgggaacacaaagcccaggaaggagaaaacagagatgtcccccagggagcacatcaagggcaaagagaccaccccctctagcctagcagtgacccagaccatggccaccaaagctcccgagtgtgtggaggacccagatatggcaaaccagaggaagactgccctggagttctgtggagagacttggagctctctctgcacattcttcctcagcatagtgcaggacacgtcatgctaa

SEQ ID NO: 3 - protein, 251 aa, Mus musculus
MRLHSLILLSFLLLATQAFSEKVRKRAKNAPHSTAEEGVEGSAPSLGKAQNKQRSRTSKSLTHGKFVTKDQATCRWAVTEEEQGISLKVQCTQADQEFSCVFAGDPTDCLKHDKDQIYWKQVARTLRKQKNICRNAKSVLKTRVCRKRFPESNLKLVNPNARGNTKPRKEKAEVSAREHNKVQEAVSTEPNRVKEDITLNPAATQTMAIRDPECLEDPDVLNQRKTALEFCGESWSSICTFFLNMLQATSC

SEQ ID NO: 4 - DNA, 756 bp, Mus musculus
atgagactccacagcctcatcctgctctccttccttctcctggctactcaggcgttctcagaaaaggtcagaaagagagccaagaacgcaccacacagcacagcggaggagggggtagagggttcagctccctcgttagggaaggcccagaataagcagagaagcaggacatctaaatctctgacgcatggcaagtttgtcaccaaagaccaagccacatgcagatgggctgtgactgaggaggagcagggcatcagcctgaaggtccagtgcacacaagccgatcaggagttttcttgtgtttttgctggtgacccaactgactgccttaaacacgacaaagaccagatctactggaaacaggttgcccgcacgctgcgcaaacagaaaaatatctgcaggaacgccaagagtgtcttgaagaccagagtgtgcagaaagagatttccagagtctaacctcaagctggtgaaccccaacgcacgtggaaacacgaagcccaggaaggagaaagcagaggtctccgcaagggagcacaacaaggtccaagaagctgtctccacggagccaaacagggtcaaagaagacatcacactcaatccagctgcgacccagaccatggccattagagatccagagtgtctagaggatccagatgtgctcaaccagaggaagaccgccctggagttctgtggggaatcttggagctccatttgcacattcttcctcaacatgttacaggcgacatcatgctaa

SEQ ID NO: 5 - DNA, 23 bp, Artificial Sequence
  forward primer for human FGF-BP
gagctctctctgcacattcttcc

SEQ ID NO: 6 - DNA, 22 bp, Artificial Sequence
  backward primer for human FGF-BP
tagcatgacgtgtcctgcacta

SEQ ID NO: 7 - DNA, 20 bp, Artificial Sequence
  forward primer for human Cyclophilin
ggaatggcaagaccagcaag

SEQ ID NO: 8 - DNA, 20 bp, Artificial Sequence
  backward primer for human Cyclophilin
ggatactgcgagcaaatggg

SEQ ID NO: 9 - DNA, 20 bp, Artificial Sequence
  forward primer for murine FGF-BP
cgttagggaaggcccagaat

SEQ ID NO: 10 - DNA, 22 bp, Artificial Sequence
  backward primer for murine FGF-BP
ttgccatgcgtcagagatttag

SEQ ID NO: 11 - DNA, 19 bp, Artificial Sequence
  forward primer for murine GAPDH
atcaacgggaagcccatca

SEQ ID NO: 12 - DNA, 20 bp, Artificial Sequence
  backward Primer for murine GAPDH
gacatactcagcaccggcct